(19) United States
(12) Patent Application Publication
(10) Pub. No.: US 2012/0088247 A1
(43) Pub. Date: Apr. 12, 2012

(54) MATERIALS AND METHODS FOR TREATING PATIENTS WITH TAXOXIFEN

(75) Inventors: David Flockhart, Indianapolis, IN (US); Zeruesenay Desta, Carmel, IN (US); Anne Nguyen, Indianapolis, IN (US); Todd Skaar, Carmel, IN (US); Lang Li, Carmel, IN (US)

(73) Assignee: Indian University Research & Technology Corporatio, Indianapolis, IN (US)

(21) Appl. No.: 13/202,418

(22) PCT Filed: Feb. 23, 2010
(86) PCT No.: PCT/US10/25109

§ 371 Date: Nov. 30, 2011

Related U.S. Application Data
(62) Division of application No. 61/154,642, filed on Feb. 23, 2009.

(51) Int. Cl. C12Q 1/68

(52) U.S. Cl. 435/6.12

(57) ABSTRACT

A pathway for the metabolism of tamoxifen is identified including the activity of at least 2 different isoforms of UGY2B7. This pathway includes isozymes of CYP3A which convert tamoxifen (TAM) to N-desmethyl-TAM, which is then converted to endoxifen by the action of CYP2D6. Once formed, endoxifen may be degraded by at least one isozyme of UGT2B7. Patients that have highly active isoforms of CYP2D6 and slow acting isoforms of UGT2B7 accumulate high levels of endoxifen and are good candidates for treatment with tamoxifen. Also disclosed are methods for screening patients with a high likelihood of benefiting from treatment with tamoxifen. These methods include testing patients for various alleles of genes known to be involved in tamoxifen metabolism and treating patients accordingly.

### PRIORITY CLAIM

This application claims the benefit of U.S. provisional patent application No. 61/154,642 filed on Feb. 23, 2009, which is incorporated herein by reference in its entirety.

### STATEMENT OF GOVERNMENT RIGHTS

Part of the development of this invention was made with government support from the National Institute of Health (NIH) under grant number GM061373. The U.S. government has certain rights in the invention.

### FIELD OF THE INVENTION

This invention relates generally to materials and methods for identifying patients that are likely to accumulate high steady state level of endoxifen when they are treated with tamoxifen and are likely to respond well when treated with tamoxifen and related compounds.

### BACKGROUND

Tamoxifen is the most widely used drug in the selective estrogen receptor modulator (SERM) class of chemotherapeutic agents. It has several indications for breast cancer that are approved by the FDA, including the treatment of all stages of breast cancer, and the prevention of breast cancer. While the drug is clearly effective, it can also cause significant adverse events that are reported to include thromboenibolic events, and a moderate increased risk of uterine cancer.

Unfortunately, both the efficacy and toxicity of tamoxifen are very unpredictable: not everyone who is treated with tamoxifen derives a benefit from the treatment, and a relatively small number of those who are treated with the drug experience serious side effects. This variability in effect is thought to be due in part to inter-individual variability in the concentration of tamoxifen and its active metabolites between patients. Given tamoxifen's utility, there exists a need for materials and methods of identifying specific patients who may benefit from treatment with the compound. Some aspects of the invention disclosed herein seek to address these needs.

### SUMMARY

Some aspects of the invention include methods of predicting the effect of treating a patient with a drug such as tamoxifen, or a pharmaceutically acceptable salt thereof, comprising the steps of: isolating a sample from a patient, wherein the sample includes a portion of the patient's DNA; and screening the sample for the alleles of a first gene, and the alleles of a second gene included in a portion of the DNA in the sample in which the first gene is CYP2D6 and the second gene is UGT2B7, and wherein the alleles of the two genes effect the metabolism of tamoxifen or a tamoxifen type compound.

Some embodiments of the invention further include the step of: correlating the alleles of CYP2D6 and the allele of UGT2B7 identified in the sample of DNA or a marker for the DNA in the sample such as mRNA or a protein with their effect on the metabolic pathway for the degradation of compounds such as tamoxifen.

In some embodiments of the invention, the alleles of CYP2D6 screened for in the sample are selected from the group consisting of: *9, *10, *17, *29, *37, *41, *45, *46, *3, *4, *5, *6, *7, *8, *11, *12, *13, *14, *15, *16, *18, *19, *20, *38, *40, *42, *44, *56, *1, *2, *33 and *35 and the alleles of the second gene UGT2B7 are selected from the group consisting of *1, and *2.

In some embodiments, the DNA or marker screening step includes contacting a sample of the DNA from the patient with a portion of a first nucleic acid that binds to at least one allele of CYP2D6 and a portion of a second nucleic acid that binds to at least one allele of UGT2B7. In some embodiments, that may be done by contacting the DNA in the sample with at least one chip, wherein the at least one chip interacts with at least one CYP2D alleles selected from the group consisting of: *1, *2, *3, *4, *5, *6, *7, *8, *9, *10, to *10AB, *11, *12, *14, *14A, *14B, *15, *17, *19, *20, *25, *26, *29, *30, *31, *35, *36, *40, and *41.

Some aspects of the invention include determining if the genotype of the patient predisposes the patient for treatment with tamoxifen, a compound such as tamoxifen, or a pharmaceutically acceptable salt of tamoxifen. These methods may include the step of assigning the alleles of CYP2D to a group designated as fully functional, wherein said fully functional alleles of CYP2D are selected from the group consisting of: *1, *2, *33 and *35. Some embodiments may include assigning alleles of CYP2D to a group designated as reduced activity, wherein said reduced activity alleles of CYP2D are selected from the group consisting of: *9, *10, *17, *29, *37, *41, *45, and *46. And some embodiments may include assigning the alleles of CYP2D to a group designated as null, wherein said null alleles of CYP2D are selected from the group consisting of: *3, *4, *5, *6, *7, *8, *11, *12, *13, *14, *15, *16, *18, *19, *20, *38, *40, *42, *44 and *56.

In some embodiments of the invention, the alleles of the UGT2B7 gene are screened for by using PCR in order to identify at least one of the alleles selected from the group consisting of: UGT2B7*1 and UGT2B7*2. Some primers that can be used to perform PCR include some primers selected from the group consisting of: SEQ. ID. No. 3, and SEQ. ID. No. 4.

Some embodiments of the invention may include a screening step that includes assaying a first set of proteins encoded by alleles CYP2D6 to determine how efficiently the first set of proteins converts tamoxifen to endoxifen and assaying a second set of proteins in the sample encoded by alleles of UGT2BY to determine how efficiently the second set of proteins degrade endoxifen.

Some aspects of the invention include assessing a patient's ability to accumulate useful steady-state levels of endoxifen when the patient is treated with, for example, about 20 mg of tamoxifen or a therapeutically acceptable salt of tamoxifen by determining the patient's genotype with respect to CYP2D6 and UGT2BY. In some embodiments, patients may be characterized or categorized as being well suited for treatment with tamoxifen if said patient is homozygous for UGT2B7*2 and has a pair of alleles for CYP2D6 selected from the group consisting of *1, *2, *33 and *35. In some embodiments the patient may be characterized as well suited for treatment with tamoxifen if said patient is heterozygous for UGT2B7*2 and UGT2B7*1 and has at least 2 alleles for CYP2D6 selected from the group consisting of *1, *2, *33 and *35. And in still other embodiments, a patient may be characterized as well suited for treatment with tamoxifen if said patient is either heterozygous or homozygous for UGT2B7*2 and UGT2B7*1 and has at least two alleles for CYP2D6 selected from the group consisting of *9, *10, *17, *29, *37, *41, *45, *46, *1, *2, *33 and *35.

In some embodiments, a patient may be characterized as being poorly suited for treatment with tamoxifen if, for example, the patient is homozygous for UGT2B7*1; and has at least two alleles for CYP2D6 selected from the group consisting of *3, *8, *11, *16, *18, *19, *20, *38, *40, *42, *44 and *56.

Still another aspect of the invention is a method of screening patients for treatment with tamoxifen, pharmaceutically acceptable salts of tamoxifen, or similar compounds comprising the steps of: dosing a patient with tamoxifen, for example, with about 20 mg of tamoxifen per day, isolating a sample from the patient and analyzing the sample to determine the amount of endoxifen present in the sample. In some embodiments, the patient may be characterized as has having a high level of endoxifen and, therefore, be likely to benefit from treatment with tamoxifen if the concentration of endoxifen measured in the sample is equal to or greater than about 50 nM. In some other embodiments, the patient may be characterized or categorized as having a low level of endoxifen and, therefore, be unlikely to benefit from treatment with tamoxifen if the concentration of endoxifen measured in the sample is equal to or less than about 25 nM. In still other embodiments the patient may be characterized or categorized as having an intermediate level of endoxifen if the concentration of endoxifen measured in the sample is greater than about 25 nM and less than about 50 nM.

Some aspects of the invention include methods of characterizing a patient, comprising the steps of: contacting a portion of a patient's DNA with a first moiety that binds to a first allele for CYPD6, a second moiety that binds to a second allele for CYPD6. a third moiety that binds to a third allele for UGT2B7, and a forth moiety that binds to a forth allele for UGT2B7. Some further embodiments include assigning a first numerical value to the first allele for CYPD6 wherein the first numerical value is, for example, 0.5 if the first allele is selected from the group consisting of: *9, *10, *17, *29, *37, *41, *45, and *46; 0 if the first allele is selected from the group consisting of: *3, *4, *5, *6, *7, *8, *11, *12, *13, *14, *15, *16, *18, *19, *20, *38, *40, *42, *44 and *56; or 1 if the first allele is selected from the group consisting of: *1, *2, *33 and *35; a second numerical value of, for example, 0.5 if the second allele is selected from the group consisting of: *9, *10, *17, *29, *37, *41, *45, and *46; 0 if the second allele is selected from the group consisting of: *3, *4, *5, *6, *7, *8, *11, *12, *13, *14, *15, *16, *18, *19, *20, *38, *40, *42, *44 and *56; or 1, for example, if the second allele is selected from the group consisting of: *1, *2, *33 and *35; assigning a third numerical value to the third allele wherein the third numerical value is, for example, 0 wherein the third allele is *1 and forth numerical value to the forth allele where in the forth numerical value is, for example, 1 wherein the forth allele is *2; adding the values of the first, the second, the third and the forth numerical values to obtain a score and determine the efficacy of tamoxifen treatment for treatment of the patient based on the score obtained by screening the patient for at least some of these alleles.

Still other aspects include methods of treating a patient with tamoxifen comprising the steps of determining if a patient is homozygous for either UG2B7*1 or for UG2B7*2, or heterozygous for these two alleles and assigning the patient to a group wherein patients homozygous UG2B7*2 are more likely to benefit from treatment with tamoxifen than are patients homozygous UG2B7*1.

### BRIEF DESCRIPTION OF THE FIGURES

FIG. 1A: Graphs illustrating that the concentration of EXE in the plasma with UGT2B7 genotype.

FIG. 1B: Graphs illustrating that the concentration of 4HT in the plasma is associated with UGT2B7 genotype.

FIG. 1C: Graphs illustrating that the concentration of TAM in the plasma is strongly associated with UGT2B7 genotype.

FIG. 1D: Graphs illustrating that the concentration of NDM in the plasma is associated with UGT2B7 genotype.

FIG. 2: Graph illustrating the association of plasma EDX concentration with different alleles of UGT2B7 and alleles of CYP2D6I with varying activities.

FIG. 3: Graphs illustrating the association between mean plasma EDX concentration and different alleles of UGT2B7 and CYP2D6 activity.

FIG. 4: Scheme illustrating a proposed metabolic pathway for the biotransformation of TAM and its metabolites.

FIGS. 5A-5P are Table 2 a summary of various known polymorphism in the CYP2D1 gene.

### DETAILED DESCRIPTION

For the purposes of promoting an understanding of the principles of the novel technology, reference will now be made to the preferred embodiments thereof, and specific language will be used to describe the same. It will, nevertheless, be understood that no limitation of the scope of the novel technology is thereby intended, such alterations, modifications, and further applications of the principles of the novel technology being contemplated as would normally occur to one skilled in the art to which the novel technology relates.

As used herein the term, “about” refers to a range of 10 percent both below and above a given value, for example, about 1.0 includes values from 0.9 to 1.1.

Tamoxifen (TAM) is a selective estrogen receptor modulator that has been used for thirty years as a mainstay in the treatment and prevention of breast cancer. Its use has diminished in recent years with the advent of the aromatase inhibitor class of drugs which have been shown to confer a 2-3% lower risk of progression free survival in the adjuvant setting where most endocrine treatment is used. However, given that the response to tamoxifen varies widely among patients, it is possible that there are cohorts of patients that will do either better or worse when treated with TAM than those treated with aromatase inhibitors. Causes of this variability may include tumor-specific variations, the cause may also include germ-line pharmacogenetic determined differences between patients. These genetic differences between patients may include differences in how tamoxifen is metabolized and transported in patients with certain genotypes as will as individual variability in the estrogen receptor signaling pathway itself.

Differential activity of enzymes involved in phase II metabolism of tamoxifen may help to explain the observed variability in patient response to tamoxifen therapy. Glucuronidation is involved in the elimination of both the first and second-generation selective estrogen receptor modulators, including tamoxifen [8] and raloxifene [9]. Tamoxifen is excreted predominantly through the bile, a process largely facilitated by tamoxifen conjugation to glucuronic acid [8], and tamoxifen glucuronides have also been identified in the urine of tamoxifen treated patients [10]. Most of the tamoxifen metabolites found in the bile of tamoxifen treated patients exist as glucuronide conjugates [8],[11] and tamoxifen metabolites are glucuronidated with high activity by human liver microsomes [12],[13]. Tamoxifen glucuronide conjugates have been identified in the serum of tamoxifen treated patients [8],[11] and it has been suggested that glucuronidation within target tissues, like the adipose tissue of the breast may also be important in terms of tamoxifen metabolism and overall tamoxifen activity [14]. Phase II metabolism of tamoxifen via sulfation has also been reported, but there are relatively little tamoxifen sulfates in urine, and our previous work has shown that the sulfotransferase (SULT) 1A1*2 polymorphism was not significantly associated with the mean concentration of tamoxifen or any of its metabolites in women treated with tamoxifen at steady state [5].

Additionally, work in this area has identified some cytochrome (CYP) genes that appear to play a role in controlling the active metabolite concentrations of tamoxifen in breast cancer patients. Some of the published alleles of the gene CYP2D6 (SEQ ID No. 1) are summarized in Table 2; see also the website www.cypalleles.ki.se/cyp2d6.

Specifically, it has been shown that tamoxifen is sequentially metabolized to its major active metabolite endoxifen by CYP3A and then CYP2D6[3] and that a patient's CYP2D6 metabolic status helps to predict not only the plasma concentrations of endoxifen in the patients, but also the patient's prognosis for progression free survival upon treatment with tamoxifen. However, variability in CYP2D6 alone does not account for all the variability observed in endoxifen concentrations and tamoxifen response. For example, high inter-subject variability in endoxifen plasma concentrations has been noted even after controlling for CYP2D6 metabolic status. The CYP2D6 genotype has been shown to be associated with clinical outcome in the adjuvant and metastatic settings in some studies, but this has not been the case in all studies and it is generally recognized that more data are needed before definitive clinical recommendations can be made concerning treatment with tamoxifen. As demonstrated herein, more refined genetic signatures are of value in making this determination.

Sulfation and glucuronidation may be involved in the elimination of tamoxifen and its metabolites from a patient. Previous work has failed to demonstrate a significant linkage between a sulfotransferase (SULT) 1A1*2 polymorphism and the mean concentration of tamoxifen or any of it's metabolites in women treated with tamoxifen. However, the SULT1A1*2 polymorphism has been shown to be associated with clinical outcome, a finding that may represent a role for this enzyme in tissue level intracellular metabolism of TAM. There is evidence that glucuronidation may be involved in the elimination of both the first and second generation selective estrogen receptor modulators, tamoxifen and raloxifene. Glucuronidation also plays a role in the elimination of endogenous estrogen, bilirubin, and some carcinogens. Glucuronidation is catalyzed by a family of dimeric microsomal enzymes called UDP-glucuronosyltransferases (UGT). Early studies have identified glucuronides of tamoxifen active metabolites such as endoxifen and 4-hydroxytamoxifen in human or animals. The specific UGT isoforms involved in these reactions have been elucidated in vitro and these results suggest that UGT2B7 is the primary catalyst in the liver for endoxifen O-glucuronidation and probably 4-hydroxytamoxifen Oglucuronidation as well. UGT2B7 (SEQ. ID No. 2) activity between human livers is variable and this may be due in part to different patients having different UGT2B7 polymorphisms. The UGT2B7*2 (His268Tyr) allele, which is present in approximately 50% of Caucasians and Asians, has been shown to reduce one activity significantly. One hypothesis consistent with these findings is that UGT2B7 variants may influence the concentrations of tamoxifen or its metabolites in the plasma of patients treated with tamoxifen.

Recent studies have characterized the specific glucuronidation pathways involved in tamoxifen metabolism and have identified the individual UGT enzymes active against tamoxifen and its major active metabolites including endoxifen and hydroxy-tamoxifen[12],[13]. Although 4-hydroxytamoxifen has been shown to be both N- and O-glucuronidated, only O-glucuronidation has been shown to occur for endoxifen [12],[13]. UGT2B7 exhibited the highest activity of any hepatic UGT against the trans isomer of both 4-hydroxy-tamoxifen and endoxifen in vitro, specifically forming their corresponding O-glucuronides[12]. In addition, a common mis-sense SNP resulting in a His>Tyr amino acid change at residue 268 of the UGT2B7 enzyme was recently associated with altered glucuronidation activity against both substrates, with the UGT2B7^{268Tyr} variant isoform (encoded by the UGT2B7*2 allele) exhibiting significantly decreased activity in both over-expressing cell lines and human liver microsomes (RS#7439366)[15]. The UGT2B7*2 allelic variant is present in approximately 50% of Caucasians and Asians; therefore, we have hypothesized that this variant may significantly alter circulating 4-hydroxy-tamoxifen and endoxifen and subsequently impact patient response to tamoxifen.

Determining the relationship between a patient's genotype and tamoxifen metabolism should enable clinicians to target the use of the drug to patients who are genetically predisposed to benefit from treatment with TAM and avoid administering it to patients that are likely to experience an adverse reaction to the compound. Various materials and methods described herein provide new methods that can be used to identify individual that will likely benefit from treatment with TAM and similar compounds.

As illustrated herein, the concentration of endoxifen, the principal active metabolite of TAM, is strongly associated with variants in both the CYP2DG and UGT2B7 genes that are thought to alter the activities of the enzymes encoded by these genes. Some genetic variants of CYPZD6 have already been associated with concentrations of the active metabolites of tamoxifen, (Gun et al., Stearns et al.) and low activity variants of CYPZD6 have also been associated with higher breast cancer recurrence rates.

As discussed herein, it is possible to test an individual patient's DNA obtained from blood, buccal swab, or saliva in order to identify genetic variants present in an individual patient that may help predict the patient's reaction to treatment with TAM. One way to use such data is to calculate a score that can be used to predict the likely effect of tamoxifen on the patient.

Some aspects of the invention disclosed herein include: (1) testing for an association of the UGT2B7*2 allele with tamoxifen and its metabolite serum concentrations in women with breast cancer taking tamoxifen; and (2) using this information to predict the approximate level of endoxifen in the patient's plasma. These data may also be used to predict the progression free survival rate of women with breast cancer being treated with tamoxifen.

### EXPERIMENTS

### Materials and Methods

Patients. The patients used for this study were selected from a prospective registry of women initiating treatment with tamoxifen in which germ line DNA and carefully annotated clinical data were archived. Subjects for the registry were recruited from the Breast Oncology Program at the University of Michigan Comprehensive Cancer Center and the Indiana University Cancer Center. Women (aged≧18 years) with newly diagnosed breast cancer who were starting a tamoxifen as standard adjuvant therapy were included in this registry. Patients were enrolled after they had completed all primary surgery, radiation, and adjuvant chemotherapy. Specimens and data from this registry has been sed for several other studies, as reported previously [5],[16],[6],[17].

Patients were excluded from the registry if they had started tamoxifen therapy concurrently with adjuvant chemotherapy, radiation therapy or other endocrine therapies. Other reasons for exclusion included current long-term corticosteroid therapy (previous use during adjuvant chemotherapy was permitted), and the use of clonidine, combinations of ergotamine and phenobarbital, or megestrol acetate for hot flash therapy. Patients who were pregnant or lactating were also excluded from the registry.

Enrolled patients were allowed to take vitamin E, selective serotonin reuptake inhibitors, or herbal remedies provided that they had been taking these drugs for at least 4 weeks and intended to continue taking them for at least the first month of participating in the study. Likewise, patients were allowed to begin therapy with the mentioned medications while participating in the study, provided that they were willing to continue the treatment for at least 1 month. The registry protocol was approved by the institutional review boards of both the Indiana University School of Medicine and the University of Michigan. All patients provided written informed consent before entry.

Study design. Reported herein are data that relate genetic polymorphisms UGT2B7 and CYP2D6 to plasma concentrations of tamoxifen and its metabolites in 220 women who had been entered into the registry. These women were selected for this study from 297 women originally enrolled in the registry because they had completed the necessary physical and laboratory examinations at baseline and 4-8 months after the start of tamoxifen therapy (20 mg/d orally in a single morning dose). At the indicated time points, medical histories, including a comprehensive list of current medications, were obtained, and blood samples (5 mL) were drawn. Given the long half-life of tamoxifen (5-7 days) [18],[19], we do not expect a large variability in plasma concentrations of tamoxifen or its metabolites at steady state as a result of differences in sampling time. Blood was collected in heparin tubes, and plasma was separated within 1 hour of blood collection by centrifugation at 2060×g. All samples (plasma and whole blood) were transferred to cryogenic vials (Corning, Cambridge, Mass.), shipped to the laboratory of the Division of Clinical Pharmacology, Indiana University, on dry ice, and stored at −80° C. until analysis.

Analysis of concentrations of tamoxifen and its metabolites in plasma. The plasma concentrations of tamoxifen and its metabolites were determined by use of an HPLC system developed and subsequently modified by our group. This method involves a column switching and online photocyclization technique in which the eluent, after chromatographic separation, passes through an ICT Beam Boost postcolumn photo-reactor supplied with a 5-m reaction coil and a 254-nm ultraviolet lamp (Advanced Separation Technologies, Whippany, N.J.), in which the photoreaction converts tamoxifen and its metabolites to highly fluorescent phenanthrene derivatives. UGT 2B7 genotyping. Genomic deoxyribonucleic acid (DNA) was extracted from the leukocyte portion of whole blood by use of a QJAamp DNA Blood Mini Kit (Qiagen, Valencia, Calif.) and used for genotyping of UGT2B7 variants. UGT2B7 codon 268 genotypes were determined by real-time PCR assays using the TaqMan Drug Metabolism Genotyping Assay C_{—}32449742_{—}20 (Applied Biosystems, Foster City, Calif.) in the ABI 7900HT sequence detection system equipped with an autoloader in the Functional Genomics Core Facility at the Penn State College of Medicine. Fifteen percent of randomly-chosen samples [including samples with each of the three potential UGT2B7 genotype groups (His268His, His268Tyr and Tyr268Tyr) as identified by real-time PCR] were further confirmed by standard PCR and cycle sequencing performed with the ABI 3130XL Capillary Sequencer at the Molecular Genetics Core Facility at the Penn State College of Medicine. The same primers were used for both PCR and sequencing: sense, 5′-CTATAGTGCTTTACTTTGACTTTTGGTTCG-3′ (SEQ ID. No. 3), located +642-+670 from the UGT2B7 translation start site); and antisense, 5′-GCTAGAAAAGCAAAGAAGGGAAAAAATGATTAGTTATATCTGA-3′ (SEQ. ID. No. 4), located +1555-+1597 from the UGT2B7 translation start site.

CYP2D6 genotyping. Genomic DNA was extracted from whole blood. CYP2D6 genotyping was conducted using two different genotyping methods: The AmpliChip CYP450 Test (Roche Diagnostics, Indianapolis, Ind.) and the Luminex CYP450C CYP2D6 assay (TM Biosciences). The AmpliChip was used for 129 of the DNA samples. It tests for 33 CYP2D6 alleles (ie, *1 to *10AB, *11, *14A, *14B, *15, *17, *19, *20, *25, *26, *29 to *31, *35, *36, *40, *41, *1N, *2xN, *4xN, *10xN, *17xN, *35xN, and *41xN) [22]. The remaining 60 samples were genotyped using the Luminex CYP450 Test. It tests for 16 CYP2D6 alleles (ie, *2 through *12, *14, *15, *17, and *41). The genotypes of approximately 10-20% of the samples were confirmed by running both assays or by using independent Taqman or restriction fragment length polymorphism (RFLP) assays.

CYP2D6 Gene Score. The CYP2D6 alleles were assigned a value that reflects the expected relative activity of the CYP2D6 enzyme for which they code. Accordingly, fully functional CYP2D6 alleles (e.g. *1, *2, *33, *35) were assigned a score of 1. CYP2D6 alleles associated with reduced enzyme activity (e.g. *9,*10, *17, *29, *37, *41, *45, *46) were scored as 0.5. The null CYP2D6 alleles (e.g.*3-*8, *11-*16, *18-*20, *38, *40, *42, *44, *56) and their duplications received a value of O. Duplications of fully active alleles (e.g. *1xN, *2xN, *35xN) were counted twice, rendering a score of 2 for each of these duplicated variants.

Duplicated reduced activity alleles (e.g. *41xN and *45xN) were assigned a value of +0.5, yielding a score of 1. For example, the score of a biallelic CYP2D6 genotype is defined as:

gene score=allele1 score+allele2 score

CYP2D6 Inhibition Factor and CYP2D6 Activity Score. After calculating the expected activity of the CYP2D6 enzyme according to the genotype, the possible effects of known CYP2D6 inhibitors on the performance of the CYP2D6 protein were incorporated. CYP2D6 inhibitors were classified as either weak or strong in accordance to the FDA definition (27) and the website www.drug-interactions.com. It was assumed that weak CYP2D6 inhibitors would decrease by half the CYP2D6 metabolic capacity in all individuals, and strong CYP2D6 inhibitors would completely abolish the CYP2D6 activity. This rule defines the CYP2D6 inhibition factor. The CYP2D6 Activity Score is expressed as the product of multiplying the CYP2D6 Gene Score by the Inhibitor factor, which is defined in the following equation: activity score=(gene score×inhibition factor),

In presenting CYP2D6 activity score data, an extensive metabolizer (EM) was defined as an individual with an activity score greater than 2; a poor metabolizer (PM) was defined with an activity score less than 1; and an intermediate metabolizer (IM) as an individual who has a score between 1 and 2. More detailed description of CYP2D6 gene and activity scores, and their comparative performance with the other CYP2D6 scores have been presented in our previous work [5].

CYP2D6/UGT2B7 combined-score: In order to combine the CYP2D6 activity score and UGT2B7 genotype, CYP2D6 was defined as 0 (PM), 1 (1M), or 2 (EM); and UGT2B7 was defined as 0 (*1/*1 or EM), 1 (*1/*2 or IM), or 2 (*2/*2 or PM). The combined score was computed by the simple addition of the two: CYP2D6 score+UGT2B7 score.

Statistical analysis. The primary hypothesis of this study was to assess the relationship between UGT2B7 genotype, CYP2D6 score, and tamoxifen metabolites. Tamoxifen metabolites (tamoxifen, 4-hydroxytamoxifen, N-desmethyl-tamoxifen, and endoxifen) were log-transformed to make them more symmetrically distributed. UGT2B7 genotypes and pharmacokinetic concentrations were examined using both a parametric method (analysis of variance) and a nonparametric method (Kruskal Willis test). The non-parametric approach was utilized because tamoxifen metabolism distribution may still have been skewed even after log-transformation. The CYP2D6/UGT2B7 combo score's correlation with tamoxifen metabolites was analyzed similarly. For all analyses, a P-value of less than 0.05 was considered statistically significant. P-values were calculated with SAS 9.1 (SAS Institute Inc., Cary, N.C.).

### Results

Demographics. A total of 220 women with breast cancer taking tamoxifen were studied. The median age was 53 years, and the mean body mass index was 28 kg/m². Most of the patients were white (94%), with a small representation of African American (4%) and biracial (0.5%) patients.

Frequency of the UGT2B7*2 allele. 57 (25.9%) patients were homozygous for the UGT2B7*2 allele (*2/*2), 109 (49.6%) patients were heterozygous (*1/*2), and 54 (24.6%) patients were homozygous for the UGT2B7*1 allele (*1/*1). The allelic frequency of UGT2B7*2 in this cohort was 50.7%, and this variant was in Hardy-Weinberg equilibrium.

Tamoxifen and Metabolite Concentration and UGT2B7 Genotype:

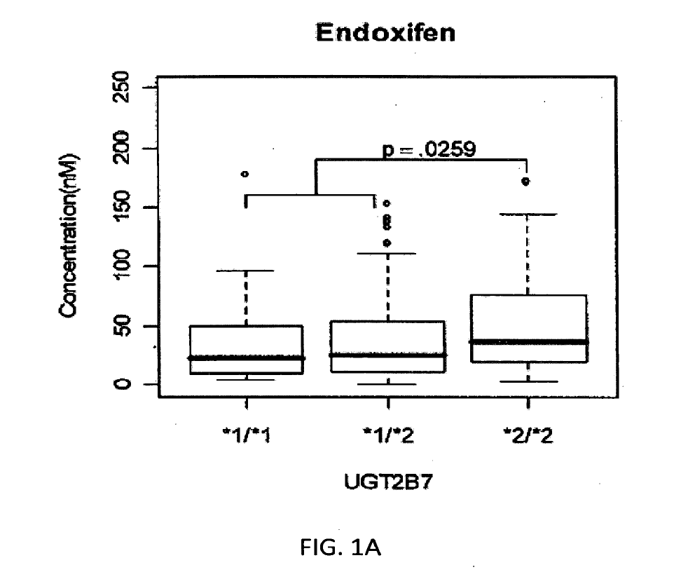

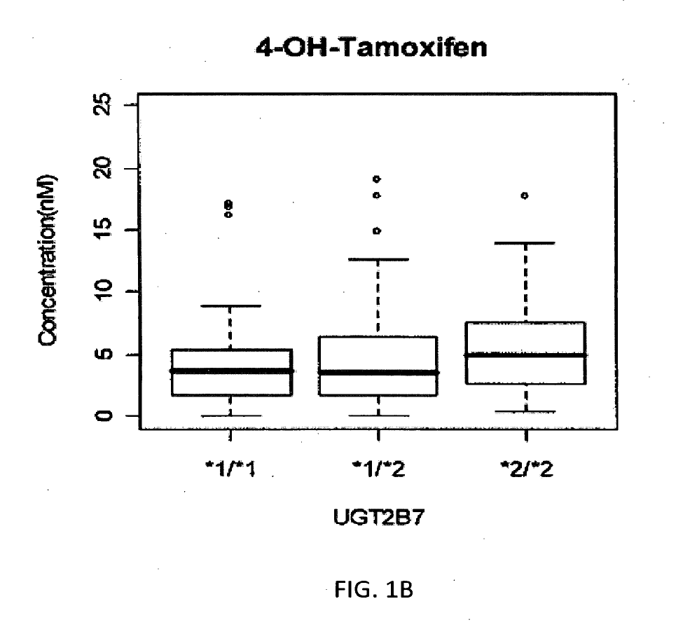

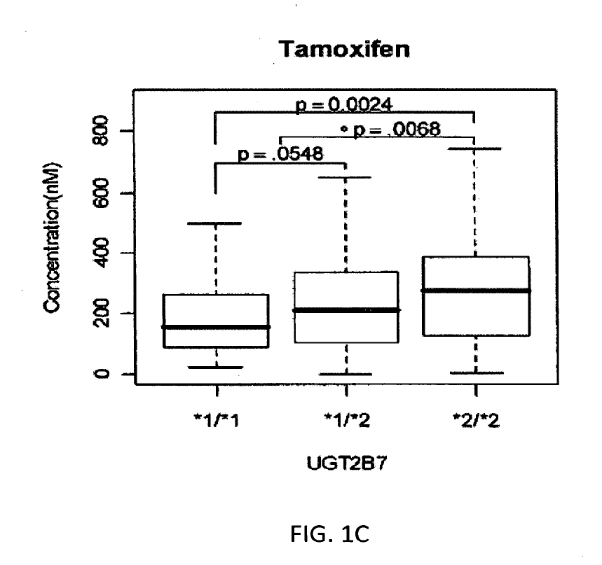

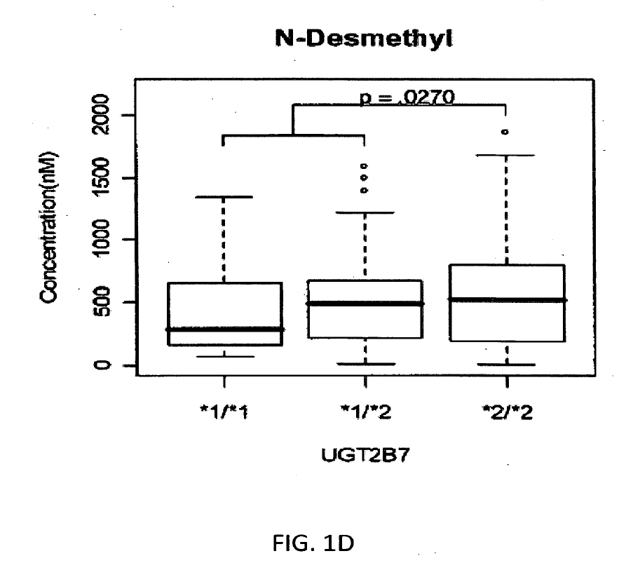

Referring now to FIG. 1. Each box plot shows the 5, 25, 50, 75 and 95 percentiles with outlying plasma concentration shown as open circles. P values are shown above the box plots. Concentration in nM is shown on the y axis. Patients were grouped into three genotype composed of UGT2B7*1/*1, *1/*2 and *2/*2 as shown on the x axis.

The associations between plasma endoxifen concentrations and UGT2B7 genotype are shown in FIG. 1. The presence of a UGT2B7 gene dose effect on mean plasma endoxifen concentration the interaction was significant (p=0.0259; FIG. 1). Still referring to FIG. 1, tamoxifen, N-desmethyl-tamoxifen and 4-hydroxy-tamoxifen plasma concentrations were not significantly associated with UGT2B7 genotype.

Mean plasma endoxifen concentration was 34% higher in the UGT2B7*2/*2 genotype group (60.3±39.9 nM) than for each of the *1/*2 (45.6±36.1 nM) and *1/*1 genotype groups (44.9±31.8 nM) (p<0.05). The mean plasma concentration of N-desmethyl-tamoxifen (FIG. 1D) was 42% larger while the mean plasma concentration of endoxifen was 47% larger (FIG. 1A) in the *2/*2 group. Mean plasma 4-OH-tamoxifen concentrations association with genotype did not reach statistical significance but did have the same trend as the other metabolites with the *2/*2 group having a 22% higher concentration than the *1/*1 group (FIG. 1B).

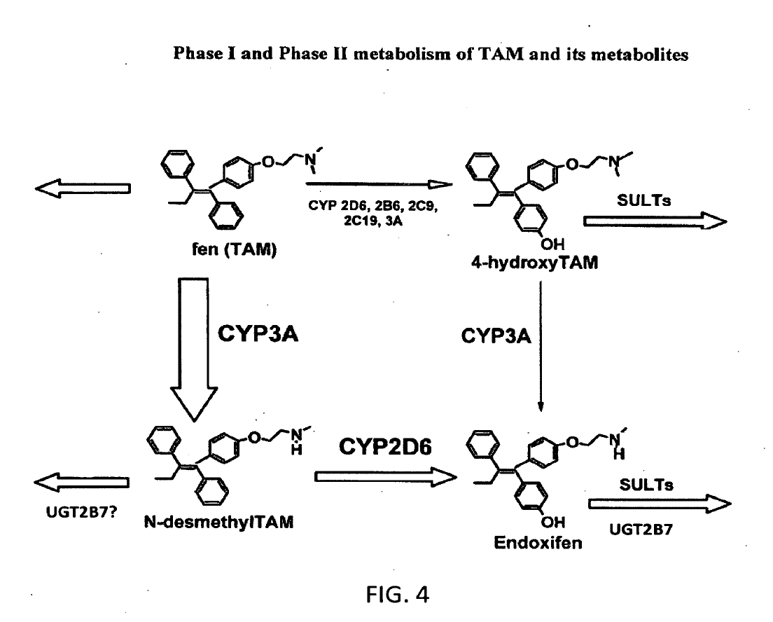

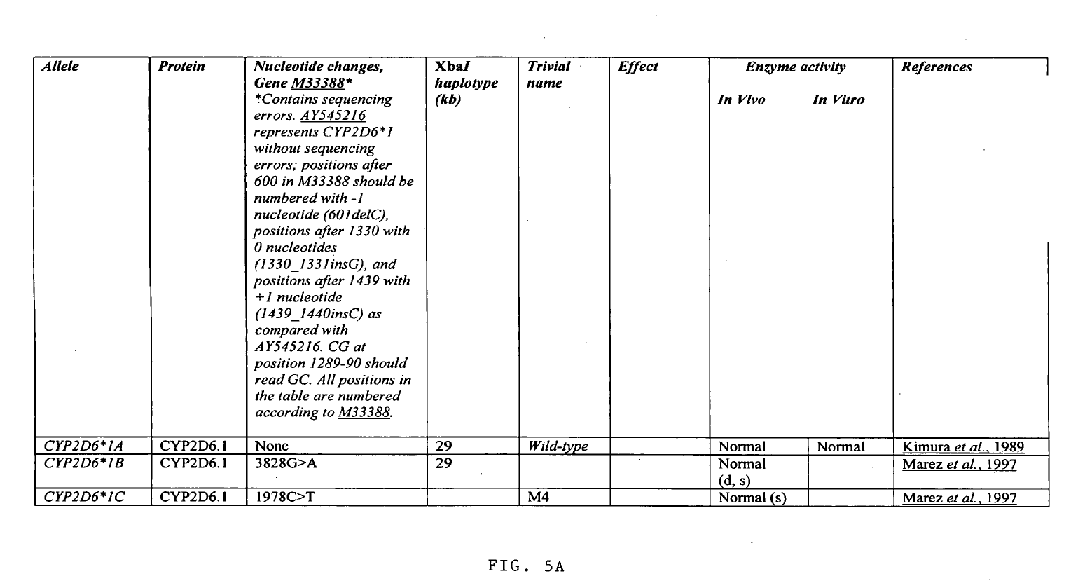

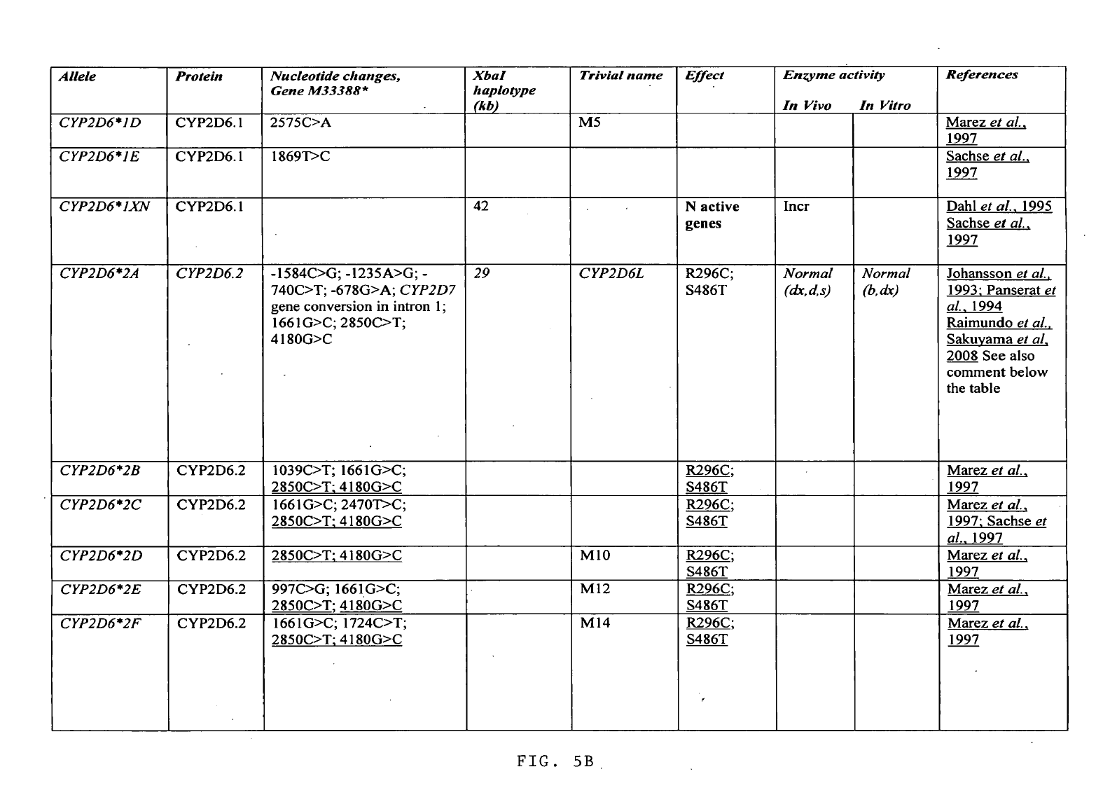

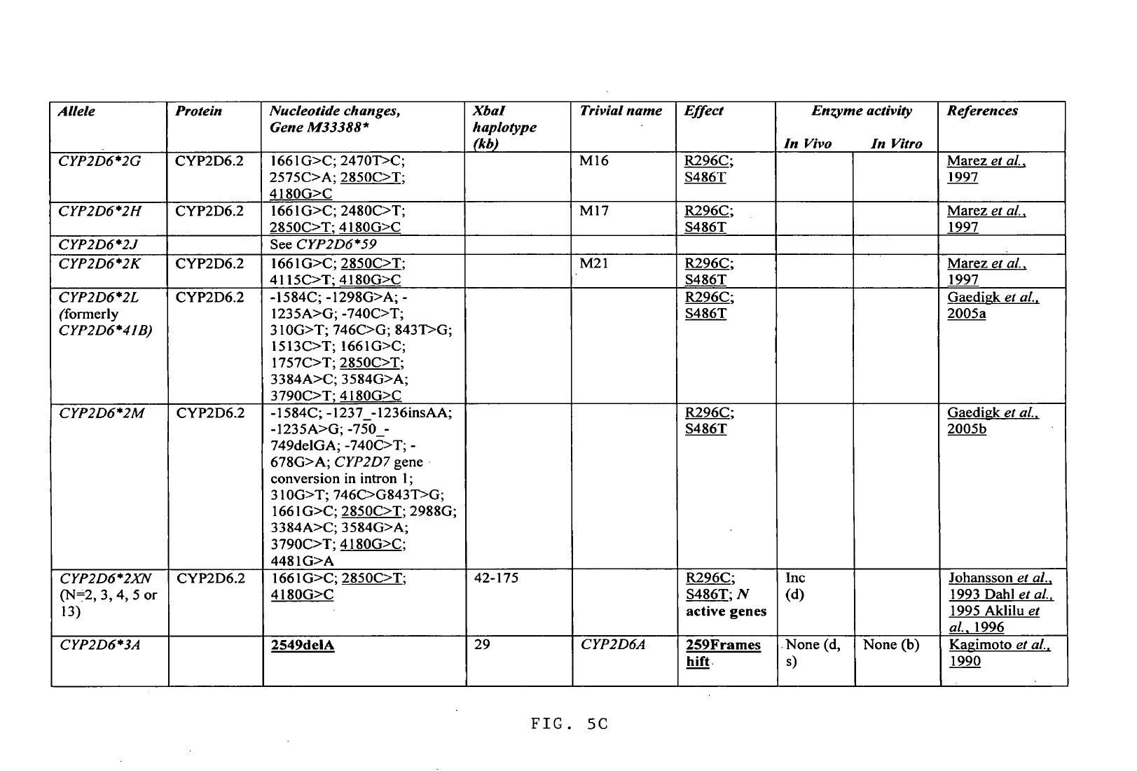

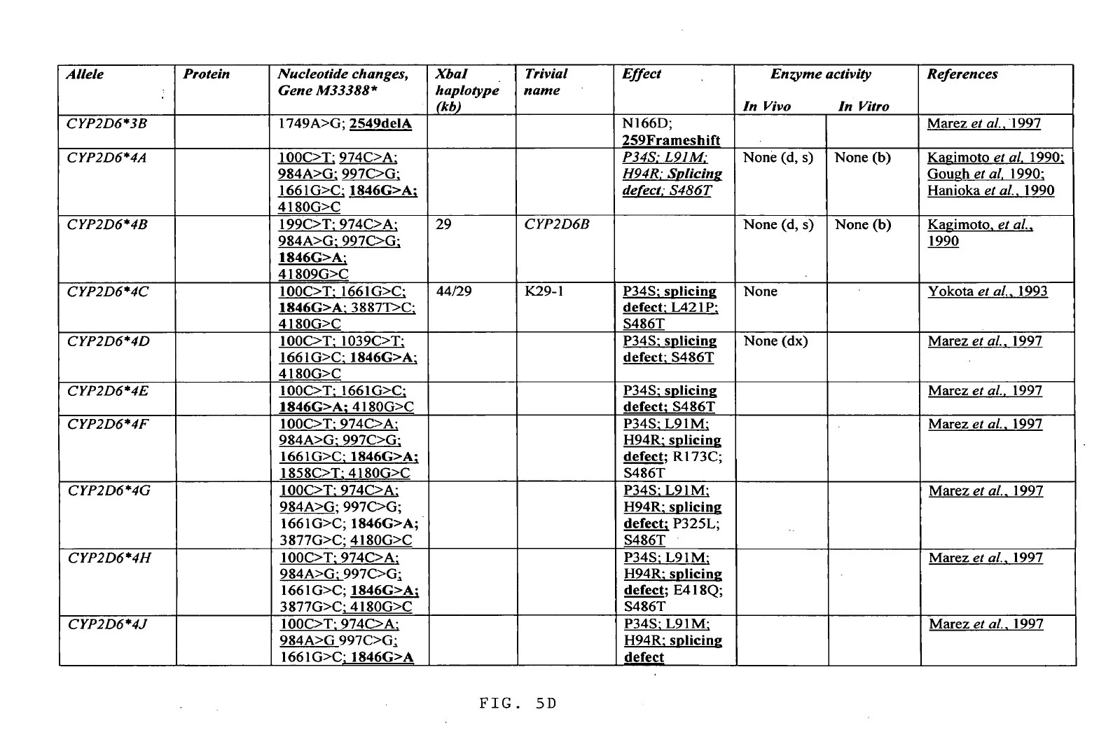

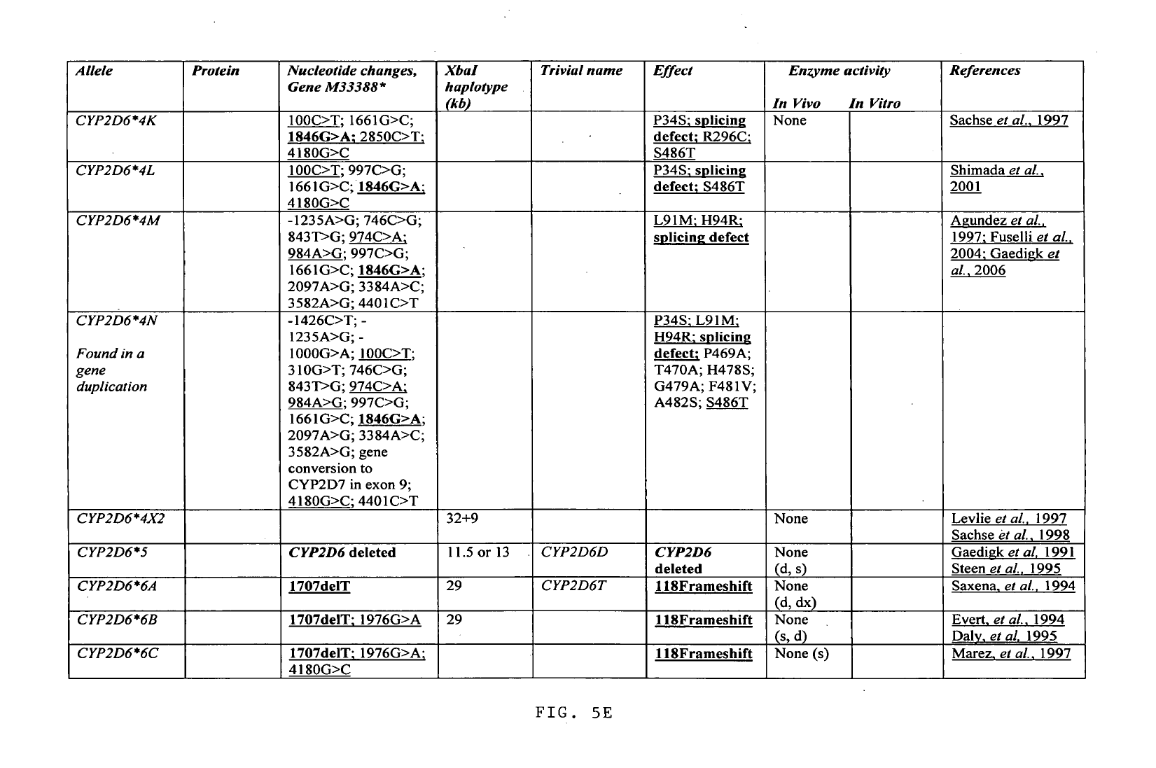

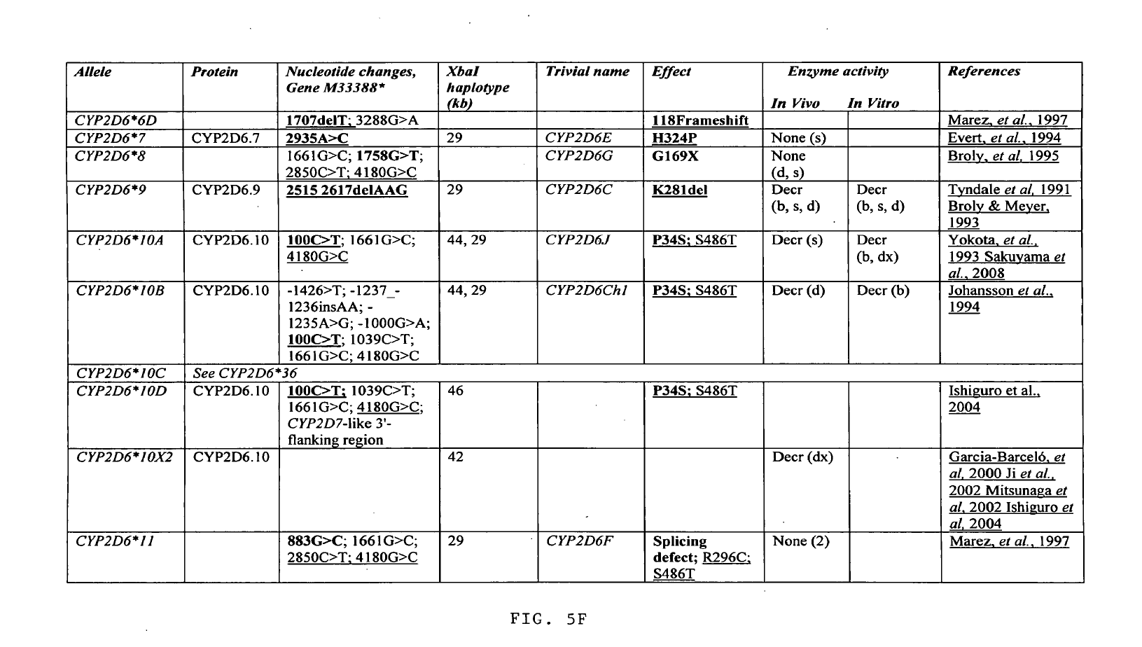

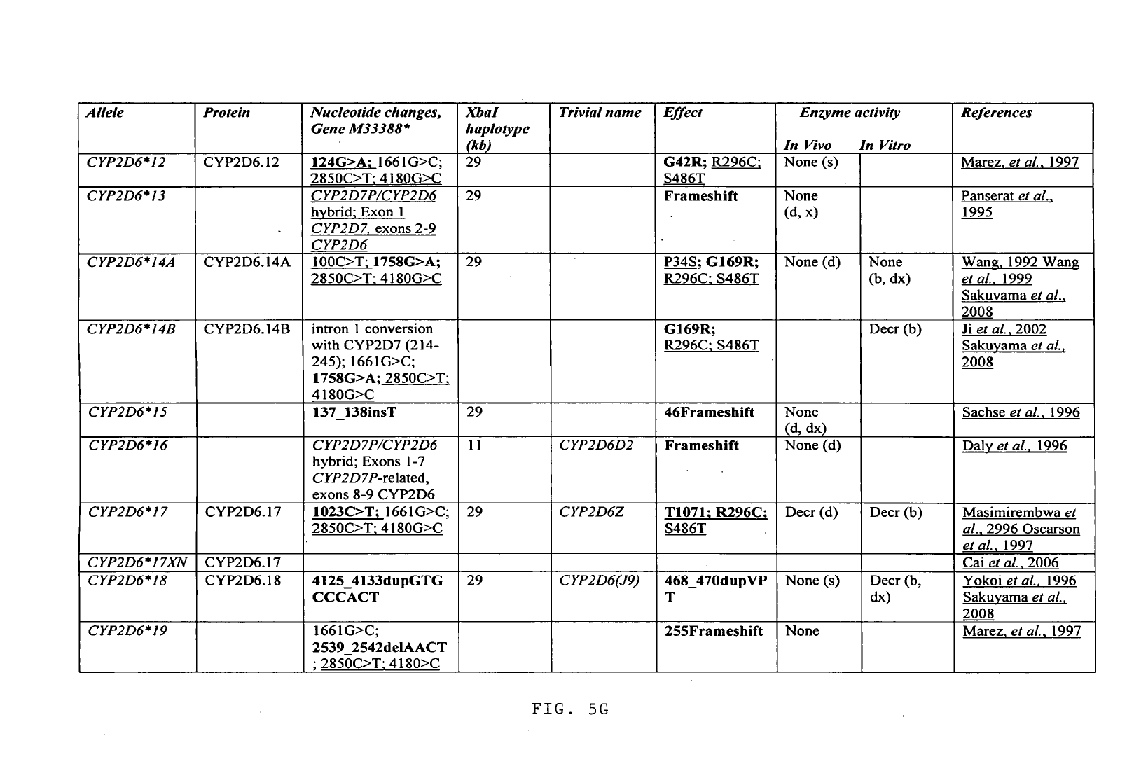

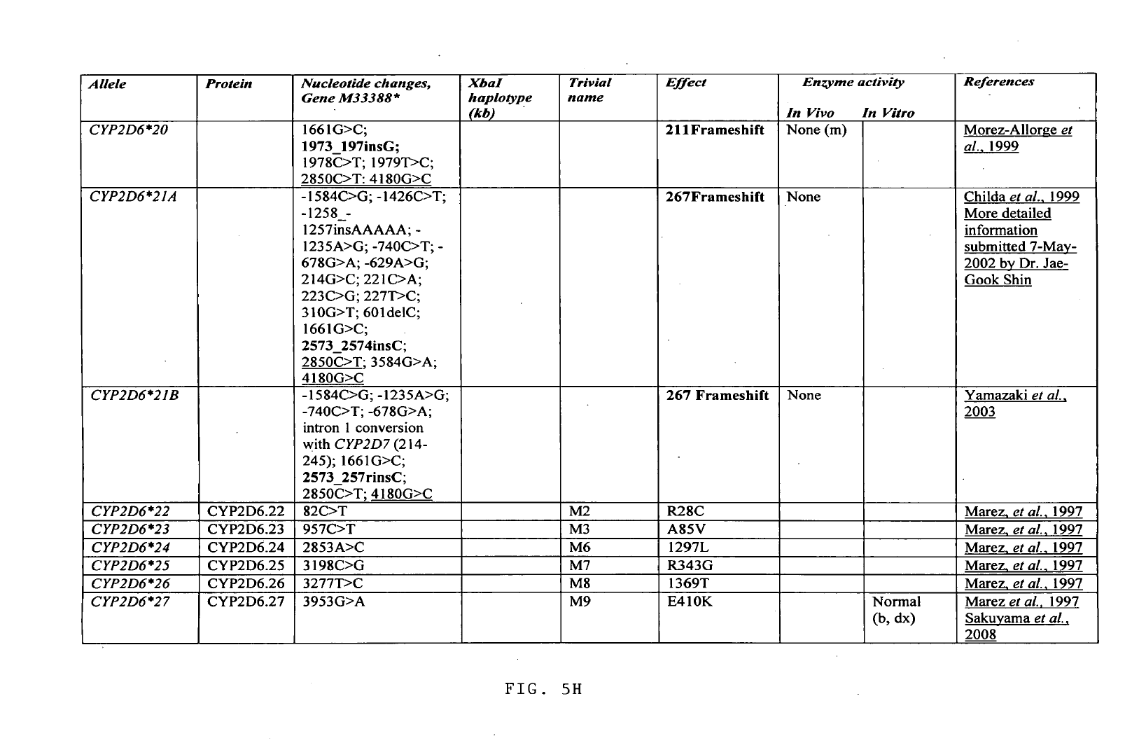

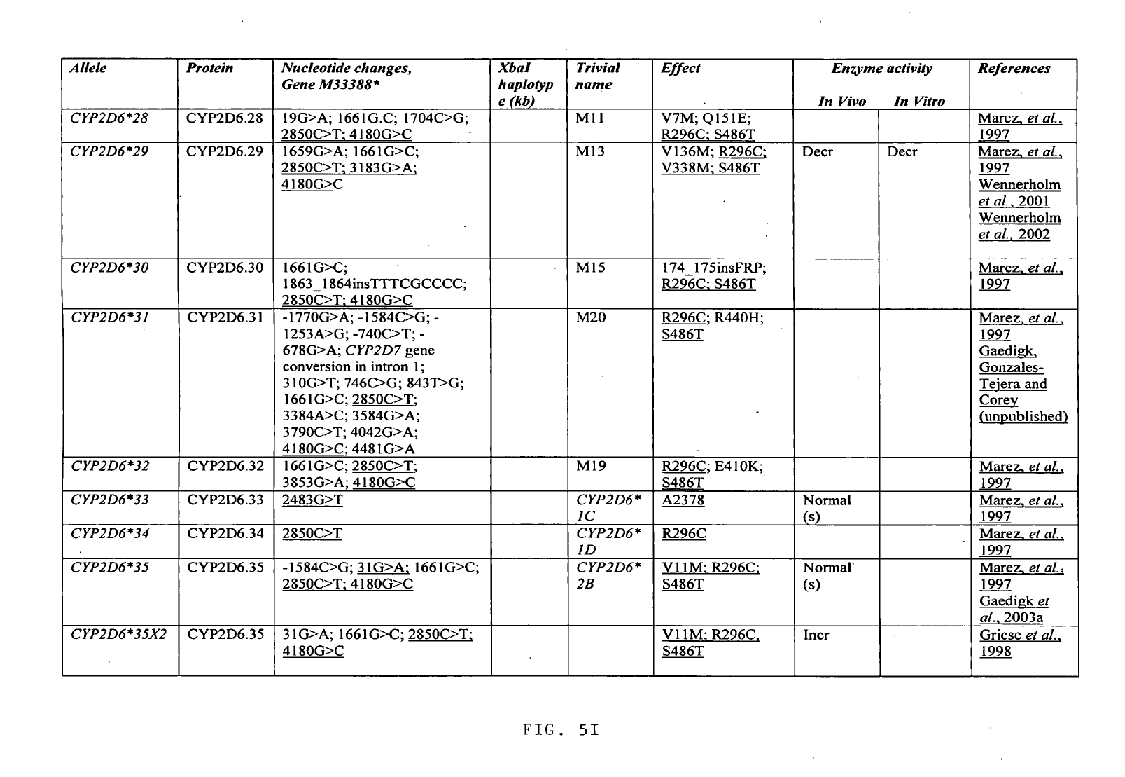

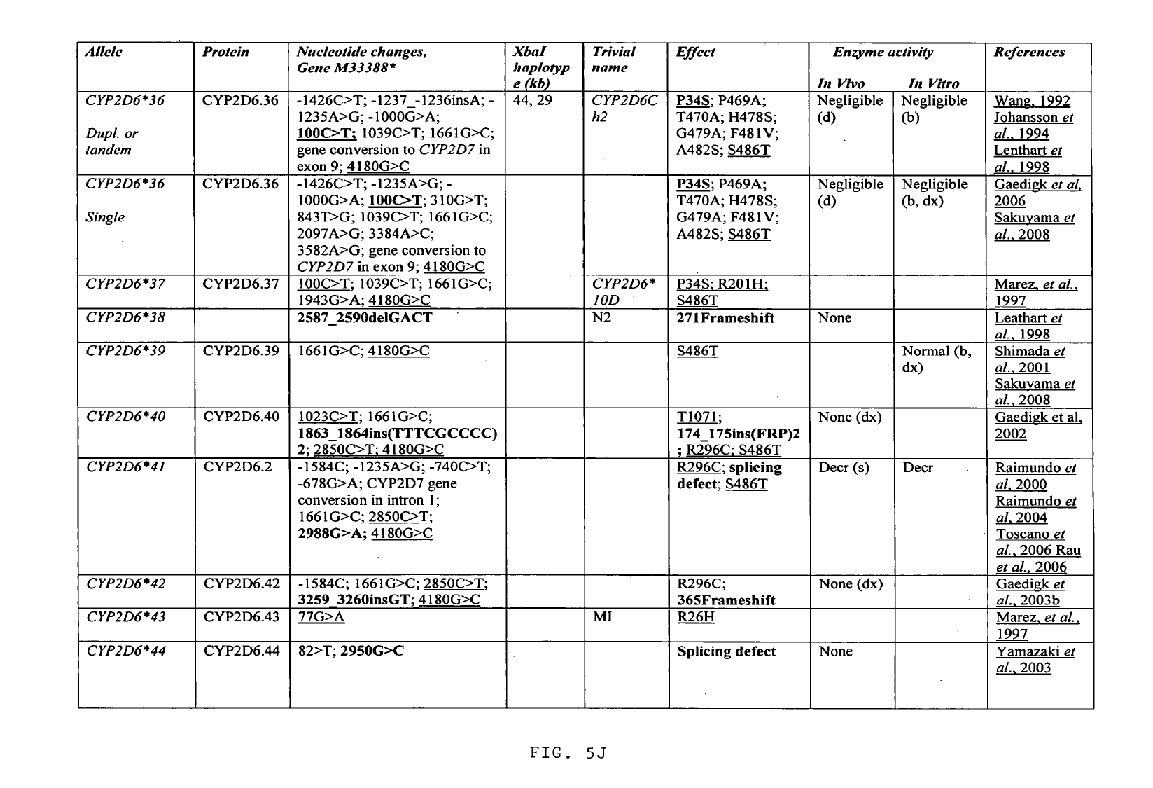

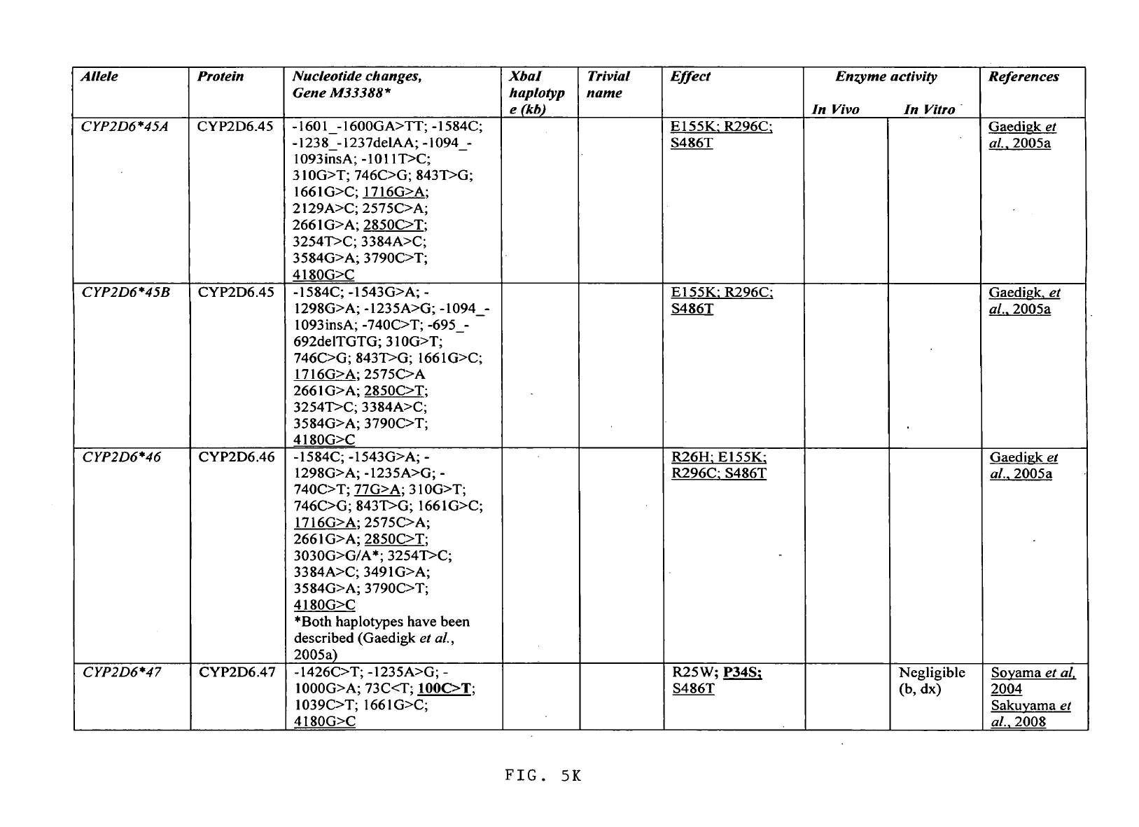

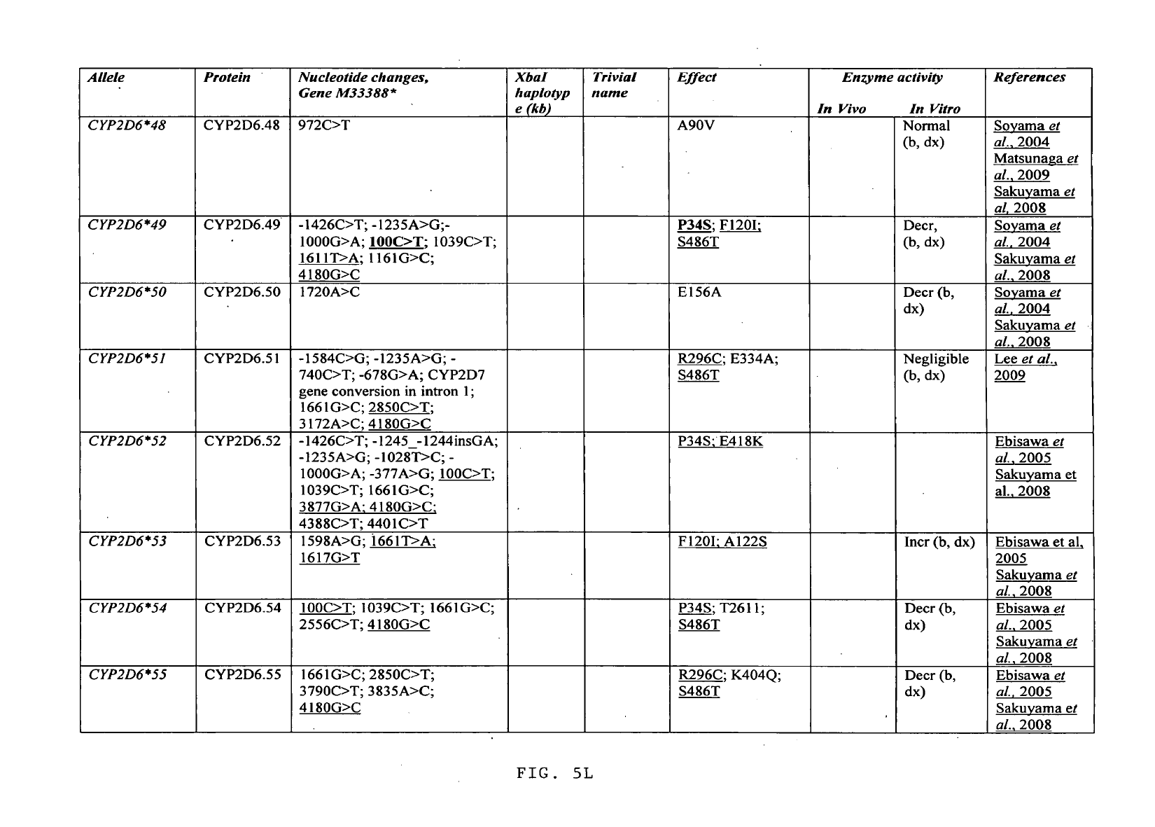

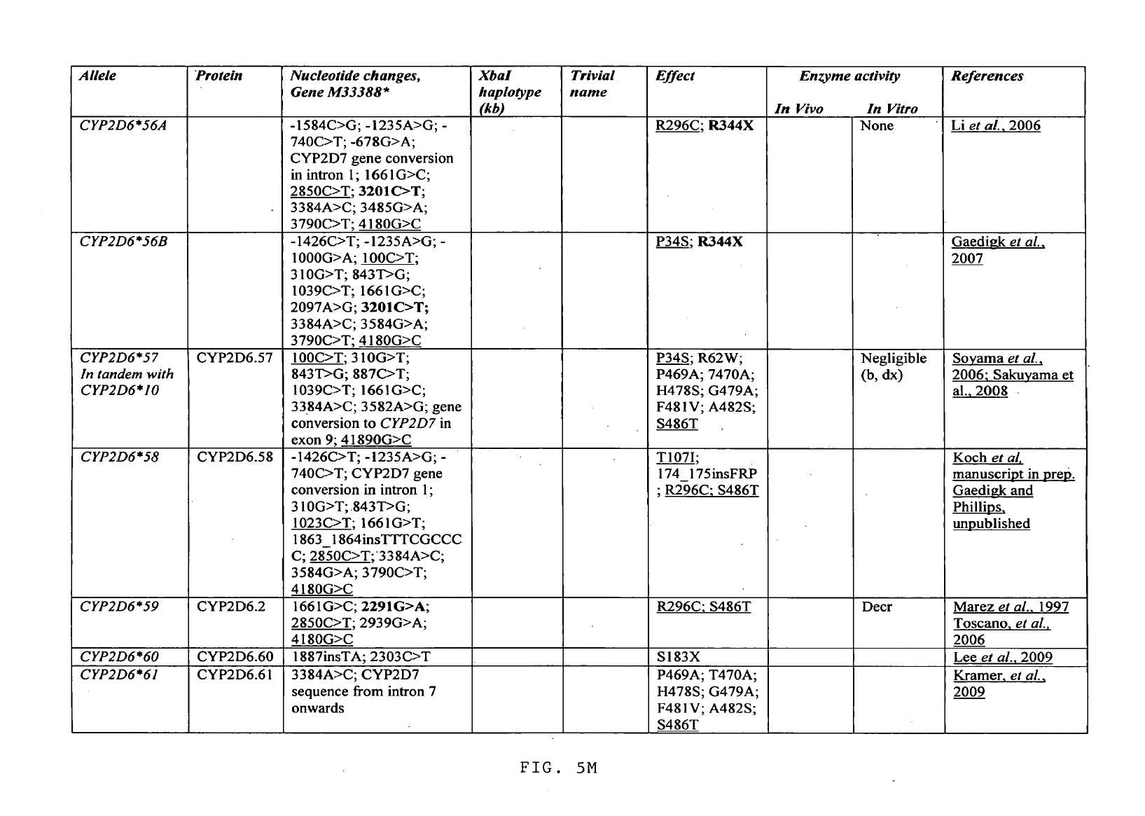

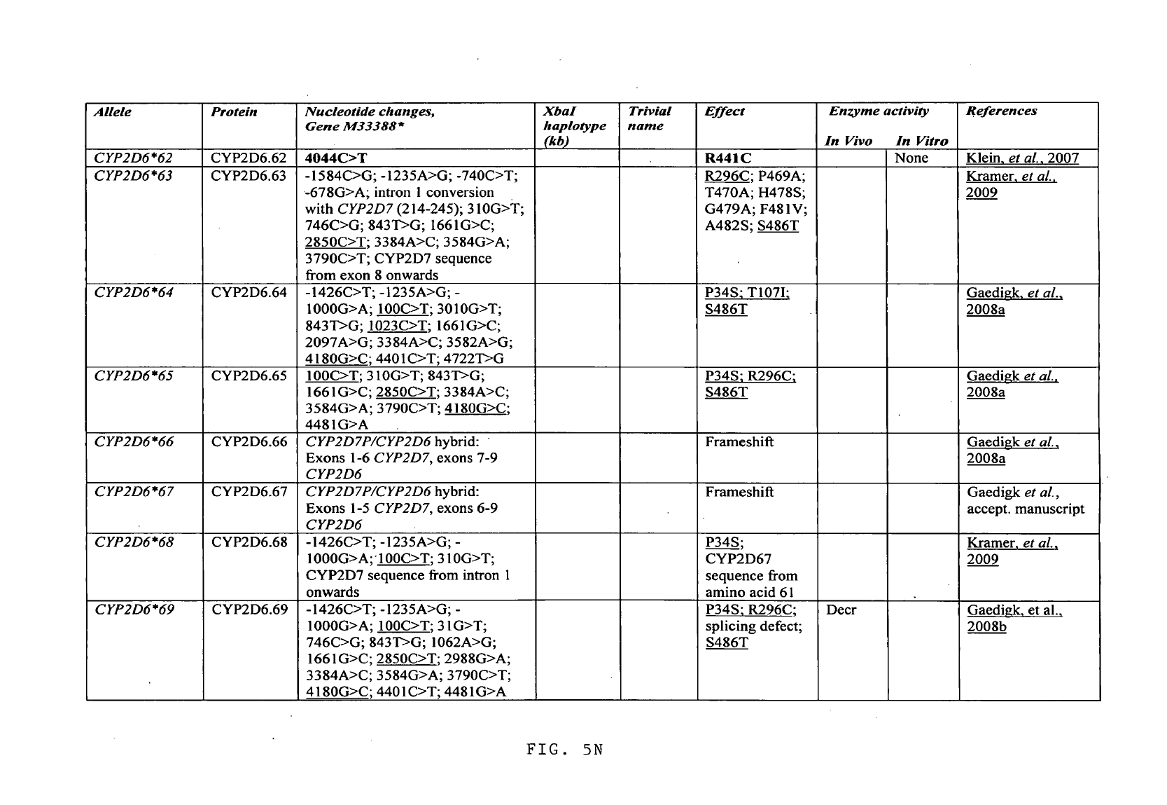

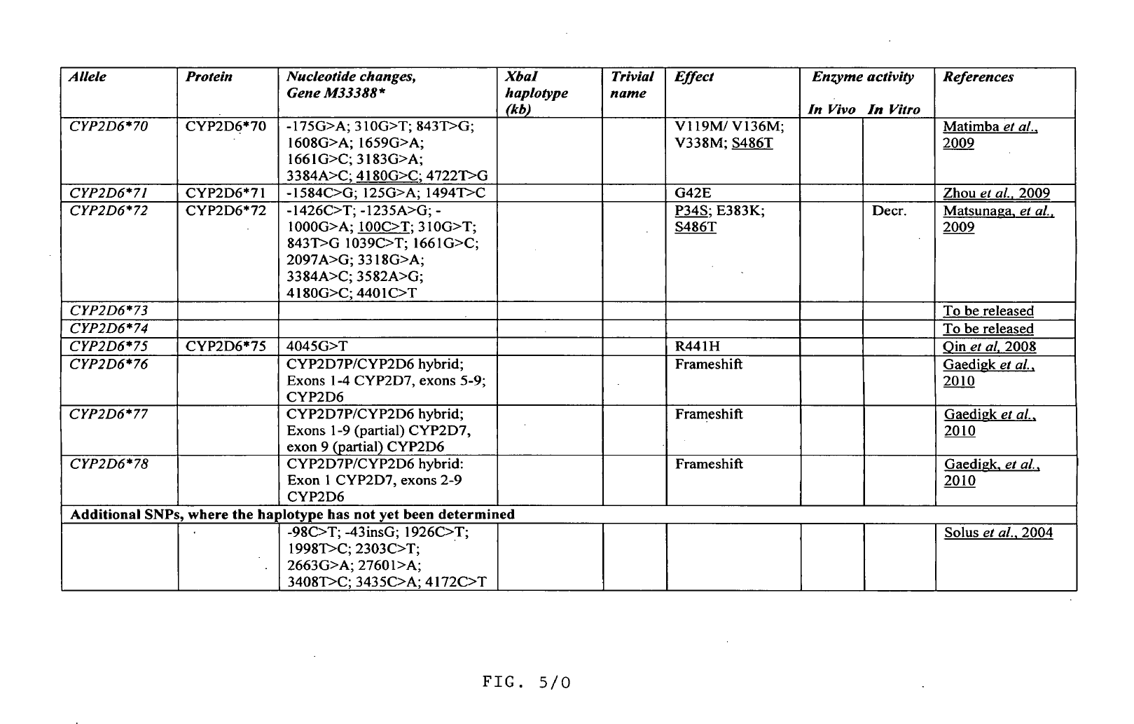

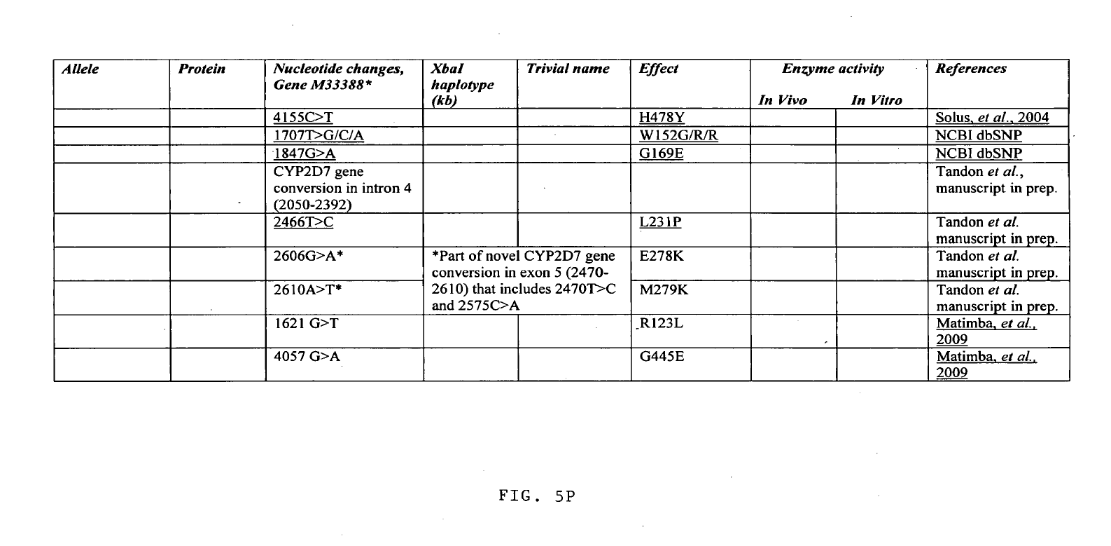

On visual inspection mean plasma concentration of both tamoxifen (FIG. 1C) and N-Desmethyl-tamoxifen (FIG. 1D) showed a gene-dose effect. But the mean plasma N-Desmethyl-tamoxifen concentration box plot (FIG. 1D) for UGT2B7*1/*2 showed the median which is slightly higher than the mean due to a right skewed distribution of the data. By visual inspection mean plasma concentration of both endoxifen (FIG. 1A) and 4-OH-tamoxifen (FIG. 1B) show the same two step trend.

Mean plasma endoxifen concentration was associated with UGT2B7 genotype but was also associated with CYP2D6 score. In the CYP2D6 extensive metabolizer group the mean plasma endoxifen concentration was strongly associated with UGT2B7 genotype but in the CYP2D6 intermediate and poor metabolizer groups mean plasma endoxifen concentration was not strongly associated with UGT2B7 genotype.

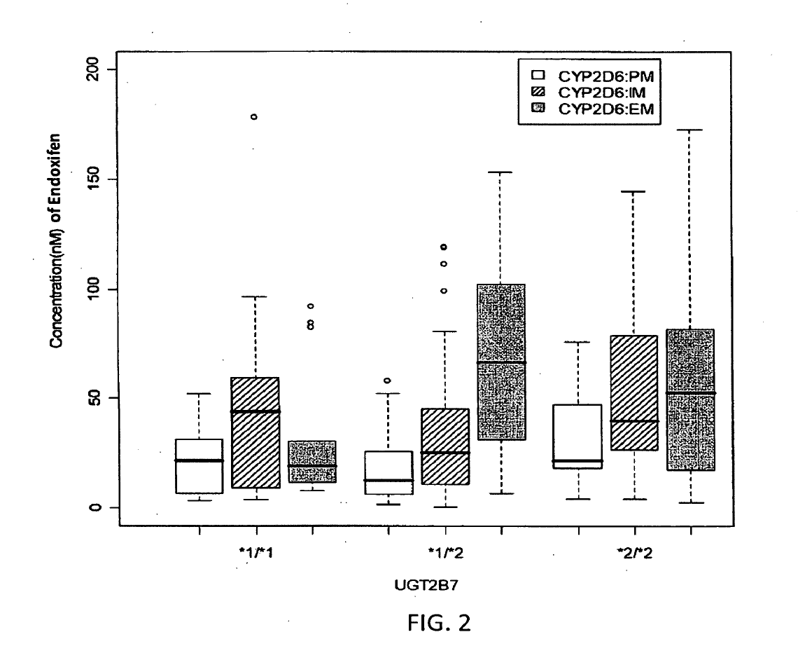

UGT2B7 genotype, as represented by its score, was able to improve the amount of variability explained by the CYP2D6 score. Table 1 shows that inclusion of UGT2B7 score improved the percent variability explained from 23% (CYP2D6 score alone) to 26% (combined score). This interaction appears to be additive. The impact of the individual and combined scores on endoxifen concentration is depicted in FIG. 2. A general linear model to evaluate the genotype-genotype interaction showed that the interaction was significant (p<0.0001). When the predicted CYP2D6 activity was high (CYP2D6 score of 2) and predicted UGT2B7 glucuronidation was low (UGT2B7 score of 2), the concentration of endoxifen was high, whereas when predicted CYP2D6 activity was low (Score of 0) and predicted UGT2B7 activity was high (Score of 0), endoxifen concentration was low. The interaction resulted in a 58% increase in endoxifen concentration in CYP2D6 extensive metabolizers with a UGT2B7*2/*2 genotype relative to CYP2D6 extensive metabolizers with a UGT2B7*1*1 genotype. When we examined the relationship between mean plasma endoxifen concentration and combined UGT2B7 genotype and CYP2D6 score, the mean plasma endoxifen concentration ranged from 19 to 107 nM. The genotype-scores ordered by rank from lowest to highest, are shown in FIG. 2. Mean plasma endoxifen concentration can now be grouped according to the combined predicted scores (0-10), as shown.

Still referring to FIG. 2. The plasma concentration of EDX associated with UGT2B7 genotype varies with CYP2D6 activity. The largest effect in EDX due to reduced UGT2B7 activity is observed in the presence of the CYP2D6 extensive metabolizers and not in the connection with either the CYP2D6 poor and intermediate metabolizers. Each box plot shows the 5, 25, 50, 75 and 95 percentiles with outlying plasma concentration shown as open circles. Patients were grouped into three genotype groups composed of UGT2B7 *1/*1, *1/*2 and *2/*2 as shown on the x axis. EDX concentration in nM is shown on the y axis. The open box plots represent the poor metabolizers, the hatched box plots represent the intermediate metabolizers and grey box plots represent the extensive metabolizers. A gene-gene interaction between the UGT2B7 sensitive CYP2D6 extensive metabolizer groups was observed. The gene-gene interaction effect on mean plasma endoxifen concentration is due to the phase one and two enzyme pharmacokinetics of the metabolic pathway. A general linear model was also used to evaluate the gene-gene interaction and found that the interaction was significant (p=0.0367). The interaction resulted in a 220% increase in concentration for CYP2D6 extensive metabolizers with a UGT2B7*2/*2 genotype.

TABLE 1
Model-based prediction of the contribution of individual and
combined activity scores of CYP2D6 and UGT2B7 on endoxifen
plasma concentration variability.

| Model | R-Square |
|---|---|
| Endoxifen = UGT2B7 score | 0.03 |
| Endoxifen = CYP2D6 score | 0.23 |
| Endoxifen = combined CYP2D6/UGT2B7 score | 0.26 |
| Endoxifen = combined CYP2D6/UGT2B7 | 0.26 |
| score + CYP2D6 * UGT2B7 | |

CYP2D6 and UGT2B7 interaction is not significant (p = 0.19)

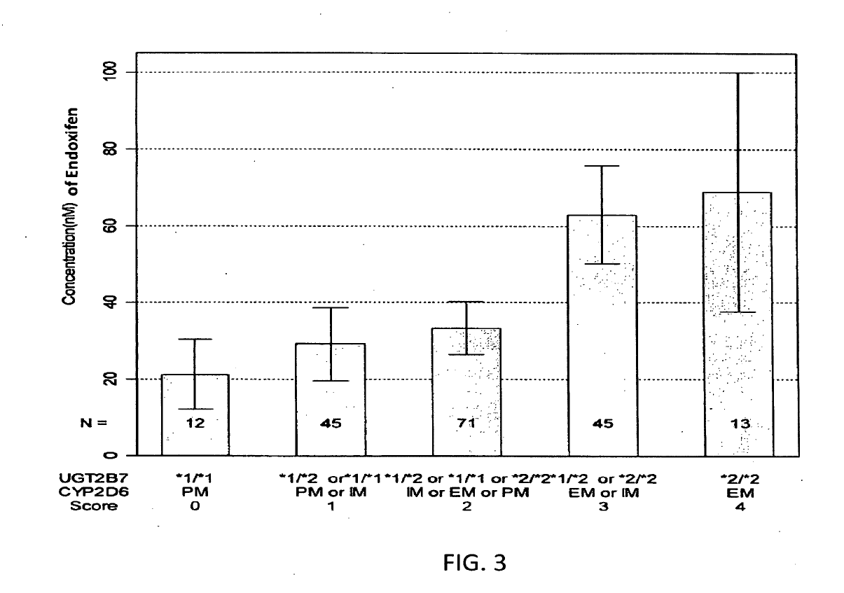

Endoxifen concentration and compound UGT 2B7 genotype and CYP 2D6 score. The relationship between mean plasma endoxifen concentration by compound UGT2B7 genotype and CYP2D6 score, mean plasma endoxifen concentration ranged from 12 to 100 nM. Referring now to FIG. 3, the compound UGT2B7 genotype and CYP2D6 score groups are ordered by compound metabolism score from lowest to highest. Each of the nine possible combinations was assigned a score based on the compound metabolism score rank order. EM, 1M and PM in UGT2B7 or CYP2D6 have been assigned a score of 2, 1, or 0 respectively. The compound metabolism score is the sum of the UGT2B7 and CYP2D6 scores. The whisker plots represent the ninety-five percent confidence intervals. The number in each bar graph is the number of patients in each compound group. Mean plasma EDX concentration in nM is shown on the y axis. UGT2B7 genotype, CYP2D6 score and rank order score are shown in the y axis. The compound genotypes are ordered by the compound metabolism score rank order from lowest to highest graphically (FIG. 3) and in tabular form (Table 3). Mean plasma endoxifen concentration by compound genotype can be grouped into the following five ranges by score.

TABLE 3
Endoxifen concentration in each (*1/*1 and PM, “*1/*2 or *1/*1”
and “PM or IM”, “1/*2 or *1/*1 or *2/*2” and “IM or EM
or PM”) are significantly different from each (“*1/*2 or *2/*2”
and “EM or IM”, *2/*2 and EM).

| UGT2B7 | CYP2D6 | [Endoxifen] nM |
|---|---|---|
| *1/*1 | PM | 21 ± 9 |
| *1/*2 or *1/*1 | PM or IM | 29 ± 10 |
| *1/*2 or *1/*1 or *2/*2 | IM or EM or PM | 33 ± 7 |
| *1/*2 or *2/*2 | EM or IM | 63 ± 13 |
| *2/*2 | EM | 69 ± 31 |

1) Score of 0. CYP2D6 poor metabolizer and no UGT2B7*2 alleles. Range of mean plasma endoxifen concentrations was 12-30 nM.

2) Score of 1. CYP2D6 poor metabolizer and one UGT2B7*2 allele or CYP2D6 intermediate metabolizer with np UGT2B7*2 alleles. Range of mean plasma endoxifen concentrations was 19 to 39 nM.

3) Score of 2. CYP2D6 extensive metabolizer and no UGT2B7*2 allele or CYP2D6 intermediate metabolizer and one UGT2B7*2 allele or CYP2D6 poor metabolizer and two UGT2B7*2 alleles. Range of mean plasma endoxifen concentrations was 26 to 40 nM.

4) Score of 3. CYP2D6 extensive metabolizer and one UGT2B7*2 allele or CYP2D6 intermediate metabolizer and two UGT2B7*2 alleles. Range of mean plasma endoxifen concentrations was 50 to 76 nM.

5) Score of 4. CYP2D6 extensive metabolizer and two UGT2B7*2 alleles. Range of mean plasma endoxifen concentrations was 38 to 100 nM.

Tamoxifen remains an important drug in the treatment of endocrinesensitive breast cancer. Although the use of aromatase inhibitors has become widespread, their benefit relative to tamoxifen is small in absolute terms. Recent data have accumulated on aromatase inhibitors demonstrated a high incidence of adverse musculoskeletal drug reactions, resulting in low compliance rates, similar to those observed with tamoxifen. It is clear that optimal treatment hinges on providing therapy that can be tolerated well and that has an optimal benefit risk ratio for individual patients. The data presented herein represent an improvement in our ability to do so.

As reported herein, there is a significant association between genetic polymorphisms in the UGT2B7 gene and plasma concentrations of tamoxifen and its metabolites. These results are consistent with other studies that showed an association between CYP2D6 genotype and active tamoxifen metabolite concentrations and progression free survival in patients with certain CYP2D6 geneotypes. Combining the effects of various UGT2B7 and CYP2D6 polymorphism on TAM metabolism, it is possible to formulate a method for identifying patients that may benefit from treatment with TAM. E.g. Patients who carry normal CYP2D6 alleles and have variants of UGT2B7 that lower the activity of this gene may derive more benefit from tamoxifen therapy than patients who carry variant alleles of CYP2D6 and normal UGT2B7 alleles. These results were used to formulate a biochemically based score to predict endoxifen concentrations. These data and the model derived there from add significantly to the understanding of the phase I and phase II metabolic pathways for tamoxifen in vivo.

Referring now to FIG. 4, CYP2D6 catalyzes the transformation of NDM to EDX, while CYP2D6, CYP2B6, CYP2C9 and CYP2C19 catalyze the transformation of TAM to 4HT. Results disclosed herein support adding N-glucuronidation by UGT2B7 to the metabolic pathway model of TAM and its metabolites. According to this model, TAM metabolism and NDM glucuronidation occurs in parallel with hydroxylation while glucuronidation of 4HT and EDX occurs in sequence with hydroxylation. The pathway depicted in FIG. 4, includes an expanded phase I and II metabolic pathway for the elimination of tamoxifen in humans. A method that takes in to account genetic variants in phase I and phase II metabolism of TAM can be used to predict the prognosis for progression free survival in patients treated with TAM and related compounds. This method was validated in a cohort of patients for whom both germline DNA data and progression free survival were data available.

Although the phase I metabolism of tamoxifen has been well studied, the influence of phase II enzymes in tamoxifen metabolism is less understood. Nishiyama, et al. suggested that trans 4-OH-tamoxifen is mainly catalyzed by sulfotransferases. However, there is scant or no evidence of a pharmacogenetic effect in vivo of SULT1A1*2 on plasma tamoxifen and metabolite concentration. There are limited in-vivo data supporting involvements of glucuronidation in 4-hydroxytamoxifen metabolism. More recently Lazarus, et al. has shown that the active metabolites 4-OH-tamoxifen and endoxifen undergo efficient O-glucuronidation by a number of UGTs including UGT2B7 in vitro. In vivo findings disclosed herein show that UGT2B7 genotype is associated with plasma endoxifen concentrations and are consistent with the in-vitro data. Surprisingly, UGT2B7 is also was associated with tamoxifen and N-desmethyl-tamoxifen plasma concentrations. These data suggest that UGT2B7 may be able to carry out N-glucuronidation of tamoxifen and N-desmethyl tamoxifen.

Previous studies have established the role CYP2D6 plays in the formation of active tamoxifen metabolites, particularly endoxifen. There is now a body of evidence suggesting that CYP2D6 influences tamoxifen outcome, probably by influencing the level of endoxifen exposure, although not all trials agree. Data presented herein strongly suggest that endoxifen, once formed by CYP2D6 is then further eliminated by UGT2B7. It is therefore possible that plasma endoxifen concentrations are much higher in people who are CYP2D6 EMs and who also carry UGT2B7 variants, because endoxifens formation by CYP2D6 is rapid and its further elimination, especially in patients with UGT2B7 variants, is relatively slow. To test this possibility, the interactions between UGT2B7 and CYP2D6 on endoxifen plasma concentrations was assessed. Using these results, it was possible to develop a combined endoxifen score was developed that includes both the CYP2D6 genotype and UGT2B7 genotype scores to estimate the total plasma endoxifen concentration. These data suggest that conversion of TAM by CYP2D6 is the concentration determining step in endoxifen levels CYP2D6 poor metabolizers, while UGT2B7 enzyme activity is more important in determining endoxifen levels in CYP2D6 extensive metabolizers.

Endoxifen plasma concentration show an association with UGT2B7 genotype which was dependent on CYP2D6 genotype as UGT2B7 dependent metabolism occurs in sequence with CYP2D6 dependent metabolism (FIG. 4). One method that can be used to predict how effectively a given patient may metabolize tamoxifen includes assigning values to each allele of these genes based on their activity. This scoring approach may also serve as a basis for accounting for the contribution of other genes that may be found to be involved in the metabolism of tamoxifen in order to continue to refine our predictions of endoxifen concentration. These methods improve the ability of clinicians to predict clinical outcomes, they will also provide powerful additional evidence demonstrating endoxifen's utility as a biomarker for TAM metabolism, and for validating of the importance of its pharmacologic role in the action of tamoxifen. While UGT2B7 was associated with tamoxifen and N-Desmethyl-tamoxifen concentrations, no gene-gene interaction was observed regarding tamoxifen and N-desmethyl-tamoxifen (data not shown). That indicates that the effect of UGT2B7 on tamoxifen and N-desmethyl-tamoxifen was independent of CYP2D6 metabolic status. This is consistent with the results illustrating that the CYP2D6 genotype shows no association with the concentrations of tamoxifen and N-desmethyl-tamoxifen.

Referring now to FIG. 4. The gene-gene interaction between phase 1 and phase 2 metabolism and endoxifen concentration is a new and highly significant finding that is consistent with the metabolic pathway proposed herein. Finding that the active metabolite concentration (endoxifen) is associated with UGT2B7 genotype is clinically significant given that in the past CYP2D6 polymorphisms with similar effect sizes were also found to effect endoxifen levels and these effects have been found to be associated with specific clinical outcomes. These results can be used to create various methods for screening patients to determine which patients my benefit from or suffer from being treated with tamoxifen. For example, a scoring system can be used to assign a value to an allele of known activity, once the alleles of these genes are known for a given patient that information may be used to determine if the patient is a good candidate for treatment with tamoxifen or a similar compound. These data also support methods for using endoxifen levels directly as a biomarker for assessing the efficacy of treating or continuing to treat a given patient with tamoxifen and similar compounds.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

### REFERENCES

1. Fisher, B., et al., Tamoxifen for the prevention of breast cancer: current status of the National Surgical Adjuvant Breast and Bowel Project P-1 study. [see comment]. Journal of the National Cancer Institute, 2005. 97(22): p. 1652-62.
2. Osborne, C. K., Tamoxifen in the treatment of breast cancer. New England Journal of Medicine, 1998. 339(22): p. 1609-18.
3. Lim, Y. C., et al., Endoxifen (4-hydroxy-N-desmethyl-tamoxifen) has anti-estrogenic effects in breast cancer cells with potency similar to 4-hydroxy-tamoxifen. Cancer Chemotherapy & Pharmacology, 2005. 55(5): p. 471-8.
4. Goetz, M. P., et al., The impact of cytochrome P450 2D6 metabolism in women receiving adjuvant tamoxifen. Breast Cancer Research & Treatment, 2007. 101(1): p. 113-21.
5. Jin, Y., et al., CYP2D6 genotype, antidepressant use, and tamoxifen metabolism during adjuvant breast cancer treatment. Journal of the National Cancer Institute, 2005. 97(1): p. 30-9.
6. Wegman, P., et al., Genetic variants of CYP3A5, CYP2D6, SULT1A1, UGT2B15 and tamoxifen response in postmenopausal patients with breast cancer. Breast Cancer Research, 2007. 9(1): p. R7.
7. Wegman, P., et al., Genotype of metabolic enzymes and the benefit of tamoxifen in postmenopausal breast cancer patients. [see comment]. Breast Cancer Research, 2005. 7(3): p. R284-90.
8. Nowell, S., et al., Association between sulfotransferase 1A1 genotype and survival of breast cancer patients receiving tamoxifen therapy. Journal of the National Cancer Institute, 2002. 94(21): p. 1635-40.
9. Lien, E. A., et al., Distribution of 4-hydroxy-N-desmethyltamoxifen and other tamoxifen metabolites in human biological fluids during tamoxifen treatment. Cancer Research, 1989. 49(8): p. 2175-83.
10. Kemp, D. C., P. W. Fan, and J. C. Stevens, Characterization of raloxifene glucuronidation in vitro: contribution of intestinal metabolism to presystemic clearance. Drug Metabolism & Disposition, 2002. 30(6): p. 694-700.
11. Swanson, C., et al., Sex steroid levels and cortical bone size in young men are associated with a uridine diphosphate glucuronosyltransferase 2B7 polymorphism (H268Y). Journal of Clinical Endocrinology & Metabolism, 2007. 92(9): p. 3697-704.
12. Hong, A. L., et al., UDP-Glucuronosyltransferase 1A1 gene polymorphisms and total bilirubin levels in an ethnically diverse cohort of women. Drug Metabolism & Disposition, 2007. 35(8): p. 1254-61.
13. Adegoke, O. J., et al., Genetic polymorphisms in uridine diphosphoglucuronosyltransferase 1A1 (UGT1A1) and risk of breast cancer. Breast Cancer Research & Treatment, 2004. 85(3): p. 239-45.
14. Sun, D., et al., Glucuronidation of active tamoxifen metabolites by the human UDP glucuronosyltransferases. Drug Metabolism & Disposition, 2007. 35(11): p. 2006-14.
15. Lonning, P. E., et al., Clinical pharmacokinetics of endocrine agents used in advanced breast cancer. Clinical Pharmacokinetics, 1992. 22(5): p. 327-58.
16. Adam, H. K., J. S. Patterson, and J. V. Kemp, Studies on the metabolism and pharmacokinetics of tamoxifen in normal volunteers. Cancer Treatment Reports, 1980. 64(6-7): p. 761-4.
17. Lee, K.-H., et al., Quantification of tamoxifen and three metabolites in plasma by highperformance liquid chromatography with fluorescence detection: application to a clinical trial. Journal of Chromatography B: Analytical Technologies in the Biomedical & Life Sciences, 2003. 791(1-2): p. 245-53.
18. Desta, Z., et al., Comprehensive evaluation of tamoxifen sequential biotransformation by the human cytochrome P450 system in vitro: prominent roles for CYP3A and CYP2D6. Journal of Pharmacology & Experimental Therapeutics, 2004. 310(3): p. 1062-75.
19. Lin, N. U. and E. P. Winer, Optimizing endocrine therapy for estrogen receptor-positive breast cancer: treating the right patients for the right length of time. [comment]. Journal of Clinical Oncology, 2008. 26(12): p. 1919-21.
20. Partridge, A. H., et al., Adherence to initial adjuvant anastrozole therapy among women with early-stage breast cancer. [see comment]. Journal of Clinical Oncology, 2008. 26(4): p. 556-62.
21. Owusu, C., et al., Predictors of tamoxifen discontinuation among older women with estrogen receptor-positive breast cancer. [see comment]. Journal of Clinical Oncology, 2008. 26(4): p. 549-55.
22. Kiyotani, K., et al., Impact of CYP2D6*10 on recurrence-free survival in breast cancer patients receiving adjuvant tamoxifen therapy. Cancer Science, 2008. 99(5): p. 995-9.
23. Nishiyama, T., et al., Reverse geometrical selectivity in glucuronidation and sulfation of cis-and trans-4-hydroxytamoxifens by human liver UDP-glucuronosyltransferases and sulfotransferases. Biochemical Pharmacology, 2002. 63(10): p. 1817-30.
24. Kakis, T., et al., Quaternary ammonium-linked glucuronidation of tamoxifen by human liver microsomes and UDP-glucuronosyltransferase 1A4. Biochemical Pharmacology, 2004. 67(11): p. 2093-102.

We claim:

1. A method of predicting the effect of treating a patient with a drug, comprising the steps of:
isolating a sample from a patient, wherein the sample includes a portion of the patient's DNA; and
screening for the alleles of a first gene, and the alleles of a second gene included in the DNA, wherein the first gene is CYP2D6 and the second gene is UGT2B7, wherein the alleles of the two genes affect the metabolism of tamoxifen or a tamoxifen type compound.

2. The method according to claim 1, further including the step of:
correlating the alleles of CYP2D6 and the allele of UGT2B7 identified in the sample with their effect on the metabolic pathway for the degradation of compounds such as tamoxifen.

3. The method according to claim 2, wherein the alleles of the first gene CYP2D6 are selected from the group consisting of: *9, *10, *17, *29, *37, *41, *45, *46, *3, *4, *5, *6, *7, *8, *11, *12, *13, *14, *15, *16, *18, *19, *20, *38, *40, *42, *44, *56, *1, *2, *33 and *35 and the alleles of the second gene UGT2B7 are selected from the group consisting of *1, and *2.

4. The method according to claim 2, wherein the screening step includes contacting a sample of the DNA from the patient with a portion of a first nucleic acid that binds to at least one allele of CYP2D6 and a portion of a second nucleic acid that binds to at least one allele of UGT2B7.

5. The method according to claim 2, wherein the screening step includes identifying the alleles of CYP2D in the sample by contacting the sample with at least one chip, wherein the at least one chip interacts with at least one CYP2D alleles selected from the group consisting of: *1, *2, *3, *4, *5, *6, *7, *8, *9, *10, to *10AB, *11, *12, *14, *14A, *14B, *15, *17, *19, *20, *25, *26, *29, *30, *31, *35, *36, *40, and *41.

6. The method according to claim 5, further including the step of assigning the alleles of CYP2D to a group designated as fully functional, wherein said fully functional alleles of CYP2D are selected from the group consisting of: *1, *2, *33 and *35.

7. The method according to claim 5, further including the step of assigning the alleles of CYP2D to a group designated as reduced activity, wherein said reduced activity alleles of CYP2D are selected from the group consisting of: *9, *10, *17, *29, *37, *41, *45, and *46.

8. The method according to claim 5, further including the step of assigning the alleles of CYP2D to a group designated as null, wherein said null alleles of CYP2D are selected from the group consisting of: *3, *4, *5, *6, *7, *8, *11, *12, *13, *14, *15, *16, *18, *19, *20, *38, *40, *42, *44 and *56.

9. The method according to claim 5, wherein the alleles of the gene UGT2B7 are screened for using PCR in order to identify at least one of the alleles selected from the group consisting of: UGT2B7*1 and UGT2B7*2.

10. The method according to claim 5, where PCR uses at least one of the primers selected from the group consisting of: SEQ. ID. No. 3, and SEQ. ID. No. 4.

11. The method according to claim 1, wherein screening step includes assaying a first set of proteins encoded by alleles CYP2D6 to determine how efficiently the first set of proteins converts tamoxifen to endoxifen and assaying a second set of proteins in the sample encoded by alleles of UGT2BY to determine how efficiently the second set of pro

12. The method according to claim 2, further including the step of:
characterizing the patient as well suited for treatment with tamoxifen if said patient is homozygous for UGT2B7*2, and has a pair of alleles for CYP2D6 selected from the group consisting of *1, *2, *33 and *35.

13. The method according to claim 2, further including the step of:
characterizing the patient as well suited for treatment with tamoxifen if said patient is heterozygous for UGT2B7*2 and UGT2B7*1 and at least 2 alleles for CYP2D6 selected from the group consisting of *1, *2, *33 and *35.

14. The method according to claim 2, further including the step of:
characterizing the patient as well suited for treatment with tamoxifen if said patient is either heterozygous or homozygous for UGT2B7*2 and UGT2B7*1 and has at least two alleles for CYP2D6 selected from the group consisting of *9, *10, *17, *29, *37, *41, *45, *46, *1, *2, *33 and *35.

15. The method according to claim 2, further including the step of:
designating a patient as poorly suited for treatment with tamoxifen if said patient is homozygous for UGT2B7*1 and has a pair of alleles for CYP2D6 selected from the group consisting of *3, *8, *11, *16, *18, *19, *20, *38, *40, *42, *44 and *56.

16. A method of screening patients for treatment with tamoxifen; comprising the steps of:
dosing a patient with tamoxifen;
isolating a sample from the patient; and
analyzing the sample to determine the amount of endoxifen present in the sample.

17. The method according to claim 14, wherein the patient is dosed with about 20 mg per day of tamoxifen.

18. The method according to claim 14, further including the step of:
characterizing the patient has having a high level of endoxifen if the concentration of endoxifen measured in the sample is equal to or greater than about 50 nM

19. The method according to claim 14, further including the step of:
characterizing the patient has having a low level of endoxifen if the concentration of endoxifen measured in the sample is equal to or less than about 25 nM.

20. The method according to claim 14, further including the step of:
characterizing the patient has having an intermediate level of endoxifen if the concentration of endoxifen measured in the sample is greater than about 25 nM and less than about 50 nM.


SEQUENCE LISTING (4 sequences)

SEQ ID NO: 1 - DNA, 9432 bp, Homo sapiens
gaattcaagaccagcctggacaacttggaagaacccggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatacaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaagaattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgcctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgagcgtggcgcatttcccagctggaatccggtgtcgaagtgggggcggggaccgcacctgtgctgtaagctcagtgtgggtggcgcggggcccgcggggtcttccctgagtgcaaaggcggtcagggtgggcagagacgaggtggggcaaagcctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcacctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctcctcagggcaaccccacccctgcctcattcctgcttaccccaccgcctggccgcatttgagacaggggtacgttgaggctgagcagatgtcagttacccttgcccataatcccatgtcccccactgacccaactctgactgcccagattggtgacaaggactacattgtcctggcatgtggggaaggggccagaatgggctgactagaggtgtcagtcagccctggatgtggtggagagggcaggactcagcctggaggcccatatttcaggcctaactcagcccaccccacatcagggacagcagtcctgccagcaccatcacaacagtcacctcccttcatatatgacaccccaaaacggaagacaaatcatggcgtcagggagctatatgccagggctacctacctcccagggctcagtcggcaggtgccagaacgttccctgggaaggccccatggaagcccaggactgagccaccaccctcagcctcgtcacctcaccacaggactggctacctctctgggccctcagggatgctgctgtacagacccctgaccagtgacgagttcgcactcagggccaggctggcgctggaggaggacacttgtttggctccaaccctaggtaccatcctcccagtagggatcaggcagggcccacaggcctgccctagggacaggagtcaaccttggacccataaggcactggggcgggcagagaaggaggaggtggcatgggcagctgagagccagagaccctgaccctagtccttgctctgccattaccccgtgtgaccccgggcccacccttccccacccttccccaccccgggcttctgtttccttctgccaacgagaaggctgcttcacctgccccgagtcctgtcttcctgctctgccttctggggctgtggcccttgctggcctggagccccaaccaagggcagggactgctgtcctccacgtctgtcctcaccgacataatgggctgggctgggcacacaggcagtgcccaagagtttctaatgagcatatgattacctgagtcctgggcagaccttcttagggaacagcctgggacagagaaccacagacactctgaggagccaccctgaggcctcttttgccagaggaccctacagcctccctggcagcagttccgccagcatttctgtaaatgccctcatgccagggtgcggcccggctgtcagcacgagagggacgttggtctgtcccctggcaccgagtcagtcagaagggtggccagggcccccttgggcccctccagagacaatccactgtggtcacacggctcggtggcaggaagtgctgttcctgcagctgtggggacagggagtgtggatgaagccaggctgggtttgtctgaagacggaggccccgaaaggtggcagcctggcctatagcagcagcaactcttggatttattggaaagattttcttcacggttctgagtcttgggggtgttagaggctcagaaccagtccagccagagctctgtcatgggcacgtagacccggtcccagggcctttgctctttgctgtcctcagaggcctctgcaaagtagaaacaggcagccttgtgagtcccctcctgggagcaaccaaccctccctctgagatgccccggggccaggtcagctgtggtgaaaggtagggatgcagccagctcagggagtggcccagagttcctgcccacccaaggaggctcccaggaaggtcaaggcacctgactcctgggctgcttccctcccctcccctccccaggtcaggaaggtgggaaagggctggggtgtctgtgaccctggcagtcactgagaagcagggtggaagcagccccctgcagcacgctgggtcagtggtcttaccagatggatacgcagcaacttccttttgaacctttttattttcctggcaggaagaagagggatccagcagtgagatcaggcaggttctgtgttgcacagacagggaaacaggctctgtccacacaaagtcggtggggccaggatgaggcccagtctgttcacacatggctgctgcctctcagctctgcacagacgtcctcgctcccctgggatggcagcttggcctgctggtcttggggttgagccagcctccagcactgcctccctgccctgctgcctcccactctgcagtgctccatggctgctcagttggacccacgctggagacgttcagtcgaagccccgggctgtccttacctcccagtctggggtacctgccacctcctgctcagcaggaatggggctaggtgcttcctcccctggggacttcacctgctctccctcctgggataagacggcagcctcctccttgggggcagcagcattcagtcctccaggtctcctgggggtcgtgacctgcaggaggaataagagggcagactgggcagaaaggccttcagagcacctcatcctcctgttctcacactggggtgtcacagtcctgggaagttcttccttttcagttgagctgtggtaaccttgtgagtttcctggagggggcctgccactacccttgggactccctgccgtgtgtctgggtctaactgagctctgaaaggagagagccccagccctgggccttccaggggaagccttacctcagaggttggcttcttcctactcttgactttgcgtctctgcagagggaggtgggaggggtgacacaaccctgacacccacactatgagtgatgagtagtcctgccccgactggcccatcctttccaggtgcagtcccccttactgtgtctgccaagggtgccagcacagccgccccactccaggggaagaggagtgccagcccttaccacctgagtgggcacagtgtagcatttattcattagcccccacactggcctgaccatctcccctgtgggctgcatgacaaggagagagaacaggctgaggtgagagctactgtcaacacctaaacctaaaaaatctataattgggctgggcagggtggctcacgcctgtaatcccagcactttgggaggccgagatgggtggatcacctgaggtcagatgttcgagaccagcctggccaacatggtgaaaccccgtctctactaaaaatacaaaaaattagctgggcgtggtggtgggtgcctgtaatcccagctactcaggaggctgaggcaggagaattgcttgaacctgggaggcagaggctgcagtgagccgagatcgcatcattgcactccagcctggtcaacaagagtgaaactgtcttaaaaaaaaaatctataattgatatctttagaaagataaaactttgcattcatgaaataagaataggagggtctaaaataaaaatgttcaaacacccaccaccactaattcttgacaaaaatatagtctgggtgccttagctcatgcctgtaatcccagcattttgggaggctaaggcaggaggattgtttgagcctaggaattc

SEQ ID NO: 2 - DNA, 1899 bp, Homo sapiens
acagaaaggaacagcaactggaaaacaagcattgcattgcaccaggatgtctgtgaaatggacttcagtaattttgctaatacaactgagcttttgctttagctctgggaattgtggaaaggtgctggtgtgggcagcagaatacagccattggatgaatataaagacaatcctggatgagcttattcagagaggtcatgaggtgactgtactggcatcttcagcttccattctttttgatcccaacaactcatccgctcttaaaattgaaatttatcccacatctttaactaaaactgagttggagaatttcatcatgcaacagattaagagatggtcagaccttccaaaagatacattttggttatatttttcacaagtacaggaaatcatgtcaatatttggtgacataactagaaagttctgtaaagatgtagtttcaaataagaaatttatgaaaaaagtacaagagtcaagatttgacgtcatttttgcagatgctatttttccctgtagtgagctgctggctgagctatttaacataccctttgtgtacagtctcagcttctctcctggctacacttttgaaaagcatagtggaggatttattttccctccttcctacgtacctgttgttatgtcagaattaactgatcaaatgactttcatggagagggtaaaaaatatgatctatgtgctttactttgacttttggttcgaaatatttgacatgaagaagtgggatcagttttatagtgaagttctaggaagacccactacattatctgagacaatggggaaagctgacgtatggcttattcgaaactcctggaattttcagtttccatatccactcttaccaaatgttgattttgttggaggactccactgcaaacctgccaaacccctgcctaaggaaatggaagactttgtacagagctctggagaaaatggtgttgtggtgttttctctggggtcaatggtcagtaacatgacagaagaaagggccaacgtaattgcatcagccctggcccagatcccacaaaaggttctgtggagatttgatgggaataaaccagataccttaggtctcaatactcggctctacaagtggataccccagaatgaccttctaggtcatccaaagaccagagcttttataactcatggtggagccaatggcatctacgaggcaatctaccatgggatccctatggtggggattccattgtttgccgatcaacctgataacattgctcacatgaaggccaggggagcagctgttagagtggacttcaacacaatgtcgagtacagacttgctgaatgcattgaagagagtaattaatgatccttcatataaagagaatgttatgaaattatcaagaattcaacatgatcaaccagtgaagcccctggatcgagcagtcttctggattgaatttgtcatgcgccacaaaggagctaaacaccttcgggttgcagcccacgacctcacctggttccagtaccactctttggatgtgattgggttcctgctggtctgtgtggcaactgtgatatttatcgtcacaaaatgttgtctgttttgtttctggaagtttgctagaaaagcaaagaagggaaaaaatgattagttatatctgagatttgaagctggaaaacctgataggtgagactacttcagtttattccagcaagaaagattgtgatgcaagatttctttcttcctgagacaaaaaaaaaaaaagaaaaaaaaatcttttcaaaatttactttgtcaaataaaaatttgtttttcagagatttaccacccagttcatggttagaaatattttgtggcaatgaagaaaacactacggaaaataaaaaataagataaagccttaaaaaaaaaaaaa

SEQ ID NO: 3 - DNA, 30 bp, Artificial Sequence
  PRIMER 1
ctatagtgctttactttgacttttggttcg

SEQ ID NO: 4 - DNA, 43 bp, Artificial Sequence
  PRIMER 2
gctagaaaagcaaagaagggaaaaaatgattagttatatctga